(19) United States
(12) Patent Application Publication
(10) Pub. No.: US 2025/0009258 A1
(43) Pub. Date: Jan. 9, 2025

(54) INFORMATION PROCESSING SYSTEM, INFORMATION PROCESSING DEVICE AND PROGRAM RECORDING MEDIUM

(71) Applicant: SEIKO EPSON CORPORATION, Tokyo (JP)

(72) Inventors: Yusuke TAKAHASHI, Matsumoto-shi (JP); Shuji UCHIDA, Shiojiri-shi (JP); Suzuyo OHARA, Chino-shi (JP)

(73) Assignee: SEIKO EPSON CORPORATION, Tokyo (JP)

(21) Appl. No.: 18/761,966

(22) Filed: Jul. 2, 2024

(30) Foreign Application Priority Data
Jul. 3, 2023 (JP) 2023-109152

(51) Int. Cl. A61B 5/11; A61B 5/00

(52) U.S. Cl. CPC A61B 5/11; A61B 5/681

(57) ABSTRACT

An information processing system includes a biological information measuring apparatus including a body unit including a first communication unit configured to communicate with another apparatus, and a first acquiring unit configured to acquire biological information of a user, and a mounting unit configured to mount the body unit on the user, and an information processing device including a second communication unit configured to communicate with the biological information measuring apparatus, and a determination unit configured to determine a part where the biological information measuring apparatus is mounted based on a movement distance from start of determination to arrival at the biological information measuring apparatus.

The present application is based on, and claims priority from JP Application Serial Number 2023-109152, filed Jul. 3, 2023, the disclosure of which is hereby incorporated by reference herein in its entirety.

### BACKGROUND

### 1. Technical Field

The present disclosure relates to an information processing system, an information processing device and a program recording medium.

### 2. Related Art

It is known to measure a biological information of a person with a wearable apparatus mounted on the body of the person.

A biological information measuring apparatus disclosed in JP-A-2003-102692 includes a sensor mounting head for measuring biological information of the user and a main body part, in which the energized pin differs for each attachment attached to the main body part, the part of the body where the main body part is mounted is determined in accordance with the energized pin, and the measurement value of the biological information is corrected in accordance with the determination result (see JP-A-2003-102692).

However, in some cases it is difficult for the known technique to specify the part of the body where the apparatus is actually mounted because of the configuration of determining the part where the apparatus is mounted by using the attachment attached to the apparatus.

### SUMMARY

An information processing system according to an aspect for solving the above-mentioned problems includes a biological information measuring apparatus that includes a body unit and a mounting unit, the body unit including a first communication unit configured to communicate with another apparatus and a first acquiring unit configured to acquire biological information of a user, and the mounting unit configured to mount the body unit on the user; and an information processing device that includes a second communication unit and a determination unit, the communication unit configured to communicate with the biological information measuring apparatus, and the determination unit configured to determine a part where the biological information measuring apparatus is mounted based on a movement distance from start of determination to arrival at the biological information measuring apparatus.

An information processing device according to an aspect for solving the above-mentioned problems includes a communication unit configured to communicate with a biological information measuring apparatus configured to acquire biological information of a user, and a determination unit configured to determine a part where the biological information measuring apparatus is mounted based on a movement distance from start of determination to arrival at the biological information measuring apparatus.

A non-transitory computer-readable storage medium according to an aspect for solving the above-mentioned problems is configured to record a program, the program causing a computer to implement a communication function to communicate with a biological information measuring apparatus configured to acquire biological information of a user, and a determination function to determine a part where the biological information measuring apparatus is mounted based on a movement distance from start of determination to arrival at the biological information measuring apparatus.

### BRIEF DESCRIPTION OF THE DRAWING

FIG. 1 is a diagram schematically illustrating a configuration example of an information processing system according to an embodiment.

FIG. 2 is a diagram illustrating a more specific configuration example of the information processing system according to the embodiment.

FIG. 3 is a diagram illustrating an example of a state where the information processing system according to the embodiment is held by a user.

FIG. 4 is a diagram illustrating an exemplary procedure of a process of determining a wearing part of a biological information measuring apparatus according to the embodiment.

FIG. 5A is a diagram illustrating a first screen display as an example of a screen display of an information processing device according to the embodiment.

FIG. 5B is a diagram illustrating a second screen display as an example of a screen display of the information processing device according to the embodiment.

FIG. 5C is a diagram illustrating a third screen display as an example of a screen display of the information processing device according to the embodiment.

FIG. 6 is a diagram schematically illustrating a calculation process for the orientation, speed and position using an IMU sensor according to the embodiment.

FIG. 7 is a table showing an exemplary relationship between a wearing position of the biological information measuring apparatus according to the embodiment and a threshold value.

FIG. 8A is a diagram illustrating an example of a state where a biological information measuring apparatus of wristwatch type according to the embodiment is mounted.

FIG. 8B is a diagram illustrating an example of an external appearance of the biological information measuring apparatus of wristwatch type according to the embodiment.

FIG. 9 is a diagram schematically illustrating a configuration example of a sensor unit of a biological information measurement device according to the embodiment.

### DESCRIPTION OF EMBODIMENTS

Embodiments are described below with reference to the accompanying drawings.

Embodiments are described below.

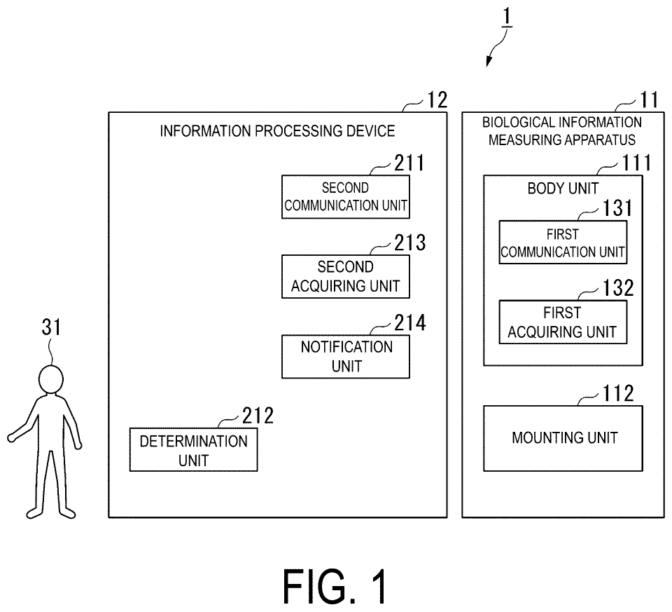

With reference to FIG. 1, an information processing system 1 according to an embodiment is briefly described.

FIG. 1 is a diagram schematically illustrating a configuration example of the information processing system 1 according to the embodiment.

The information processing system 1 includes a biological information measuring apparatus 11 and an information processing device 12.

In addition, FIG. 1 illustrates a user 31 for convenience of description. The user 31 is a living body, and is a human being in the embodiment.

In the embodiment, the biological information measuring apparatus 11 is a wearable apparatus that is mounted on the user 31, and the information processing device 12 is a smartphone owned by the user 31.

The biological information measuring apparatus 11 may be owned by the user 31, or temporarily lent to the user 31, for example.

Likewise, the information processing device 12 may be owned by the user 31, or temporarily lent to the user 31, for example.

Note that the information processing device 12 may be a mobile apparatus other than smartphones, for example.

The biological information measuring apparatus 11 is described below.

The biological information measuring apparatus 11 includes a body unit 111 and a mounting unit 112.

The body unit 111 includes a first communication unit 131 and a first acquiring unit 132.

The first communication unit 131 has a function to communicate with another apparatuses. The first communication unit 131 has a function to perform short-range wireless communication, for example. In the embodiment, another apparatus includes at least the information processing device 12.

The first acquiring unit 132 has a function to acquire the biological information of the user 31.

Various information related to a living body may be used as the biological information of the user 31.

For example, information about the physical quantity itself representing the movement of the user 31 may be used, or other information obtained based on such a physical quantity may be used as the biological information of the user 31, for example. The movement of the user 31 may be a movement of head, chest, abdomen, arms, elbows, hands, legs, knees, or feet of the user 31, or a movement of the heart, lungs, or blood inside the user 31.

Examples of the information about the physical quantity itself representing the movement of the user 31 include the acceleration of a predetermined portion of the user 31, the angular velocity of the rotation of a predetermined portion of the user 31, the temperature of a predetermined portion of the user 31, electric signals generated from the user 31, and magnetic signals generated from the user 31.

In addition, examples of other information obtained based on the above-mentioned physical quantity include the number of steps walked by the user 31, the heart rate of the user 31, the pulse rate of the user 31, or the blood pressure of the user 31.

In addition, as the biological information of the user 31, information other than the movement of the user 31, such as the body fat percentage of the user 31, may be used, for example.

The mounting unit 112 is a component for mounting the body unit 111 on the user 31.

As the mounting unit 112, various mounting configurations such as bands, belts, clothing, shoes, hats, helmets, and accessories may be used.

As the biological information measuring apparatus 11, various wearable apparatuses may be used.

For example, the part of the user 31 where the biological information measuring apparatus 11 is mounted may be various parts such as the head, chest, abdomen, arms, elbows, hands, legs, knees, and feet. In addition, for each part, the left part and right part may be distinguished, and the center part may also be distinguished. Examples of the left part and right part may be the left eye and right eye, the left ear and right ear, the left chest and right chest, the left abdomen and right abdomen, the left arm and right arm, the left elbow and right elbow, the left hand and right hand, the left leg and right leg, the left knee and right knee, and the left feet and right feet.

For example, as a sensor provided in the biological information measuring apparatus 11, a sensor for detecting various information related to a living body may be used.

The information processing device 12 is described below.

The information processing device 12 includes a second communication unit 211, a determination unit 212, a second acquiring unit 213, and a notification unit 214.

The second communication unit 211 has a function to communicate with the biological information measuring apparatus 11. The second communication unit 211 has a function to perform short-range wireless communication, for example.

The determination unit 212 has a function to determine the part where the biological information measuring apparatus 11 is mounted based on the movement distance of the information processing device 12 from the start of determination to the arrival at the biological information measuring apparatus 11.

The position where the information processing device 12 is present at the start of determination may be referred to as the position of the start point or the like, for example.

In addition, the position where the information processing device 12 is present when the information processing device 12 reaches the biological information measuring apparatus 11 may be referred to as the position of the end point or the like, for example.

In addition, in the embodiment, the short-range wireless communication between the biological information measuring apparatus 11 and the information processing device 12 is also referred to as near field communication for convenience of description.

In the embodiment, NFC is used as the near field communication.

Note that the radio communication between the information processing device 12 and the biological information measuring apparatus 11 may be performed by other communication schemes, such as Bluetooth (registered trademark) LE, and Wi-Fi. That is, the radio communication scheme of the first communication unit 131 and the second communication unit 211 is not limited, and schemes that enables communication when the distance between the biological information measuring apparatus 11 and the information processing device 12 is small are used, for example.

In the embodiment, the first communication unit 131 of the biological information measuring apparatus 11 and the second communication unit 211 of the information processing device 12 perform the short-range wireless communication.

In the embodiment, the determination unit 212 determines that the information processing device 12 has reached the biological information measuring apparatus 11 through the short-range wireless communication performed by the first communication unit 131 and the second communication unit 211.

In addition, the determination unit 212 may detect the rotational direction of the information processing device 12 that is being moved to detect the part where the biological information measuring apparatus 11 is mounted based on the movement distance and rotational direction of the information processing device 12.

The second acquiring unit 213 has a function to acquire information related to the height of the user 31.

When the information related to the height of the user 31 is acquired by the second acquiring unit 213, the determination unit 212 may use the height of the user 31 when determining the part where the biological information measuring apparatus 11 is mounted.

As an example, the determination unit 212 may detect the part where the biological information measuring apparatus 11 is mounted based on the height of the user 31, the movement distance and the rotational direction.

As another example, the determination unit 212 may detect the part where the biological information measuring apparatus 11 is mounted based on the height of the user 31 and the movement distance.

Note that when the information related to the height of the user 31 is not used, the second acquiring unit 213 may not be provided in the information processing device 12.

Various methods may be used as the method of determining the part where the biological information measuring apparatus 11 is mounted with the determination unit 212.

For example, the determination unit 212 may determine that the part where the biological information measuring apparatus 11 is mounted is an arm when the movement distance is smaller than a predetermined distance, and may determine that the part where the biological information measuring apparatus 11 is mounted is below the knee when the movement distance is equal to or greater than the predetermined distance.

The first acquiring unit 132 of the biological information measuring apparatus 11 has a function to reflect on the acquired biological information a correction value corresponding to the determination result of the determination unit 212 of the information processing device 12.

Specifically, in the biological information measuring apparatus 11, when performing a predetermined process based on the biological information acquired by the first acquiring unit 132, the predetermined process can be adapted to the part where the biological information measuring apparatus 11 is mounted by reflecting a correction value on a parameter or the like of the predetermined process in accordance with the part. As the parameter, a threshold value may be used, for example.

As the process of reflecting a correction value on a parameter and the like, a process of calculating the parameter after the correction and the like may be used, or a process of selecting, as the parameter after the correction, one of a plurality of candidate preset values that are stored in advance may be used, for example.

Regarding the threshold value that is an example of the correction value, the first acquiring unit 132 of the biological information measuring apparatus 11 may use a configuration in which a first threshold value for the strength of the movement in the case where the part where the biological information measuring apparatus 11 is mounted is an arm is smaller than a second threshold value for the strength of the movement in the case where the part where the biological information measuring apparatus 11 is mounted is below the knee.

Regarding the threshold value that is an example of the correction value, the first acquiring unit 132 of the biological information measuring apparatus 11 may use a configuration in which a third threshold value for the occurrence frequency of the movement in the case where the part where the biological information measuring apparatus 11 is mounted is an arm is greater than a fourth threshold value for the occurrence frequency of the movement in the case where the part where the biological information measuring apparatus 11 is mounted is below the knee.

The notification unit 214 makes a notification of the part where the biological information measuring apparatus 11 is mounted determined by the determination unit 212.

Various methods such as a notification method using a screen display and a notification method using sound output may be used as the method of the notification of the notification unit 214.

Note that when such a notification is not made, the notification unit 214 may not be provided in the information processing device 12.

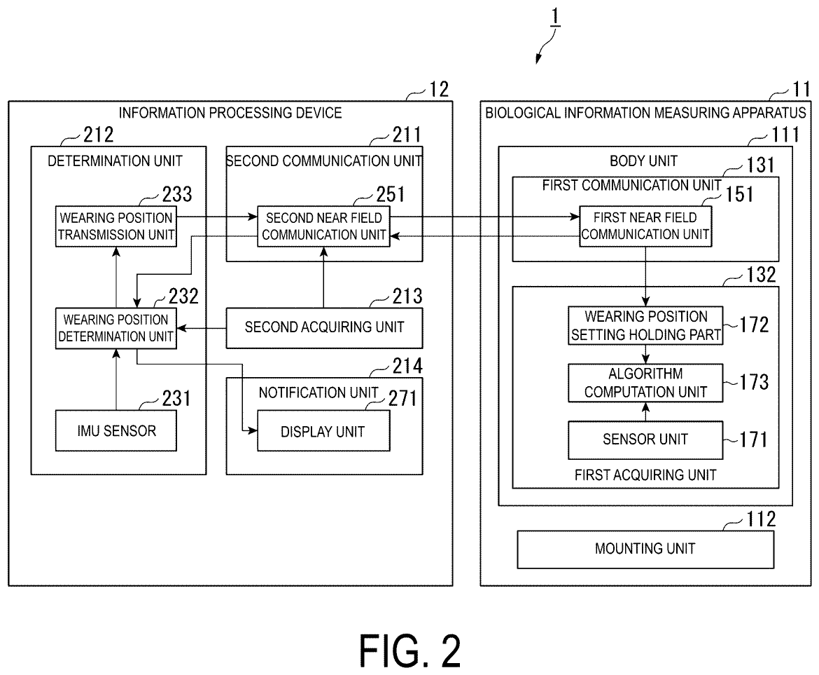

Next, a more specific configuration example of the information processing system 1 is described with reference to FIG. 2. FIG. 2 is a diagram illustrating a more specific configuration example of the information processing system 1 according to the embodiment.

The configuration example illustrated in FIG. 2 is a more specific configuration example of the configuration example illustrated in FIG. 1.

The first communication unit 131 of the biological information measuring apparatus 11 includes a first near field communication unit 151. Note that the first communication unit 131 corresponds to the first near field communication unit 151 when it includes no communication function other than the first near field communication unit 151.

The first acquiring unit 132 of the biological information measuring apparatus 11 includes a sensor unit 171, a wearing position setting holding part 172, and an algorithm computation unit 173.

The second communication unit 211 of the information processing device 12 includes a second near field communication unit 251. Note that the second communication unit 211 corresponds to the second near field communication unit 251 when it includes no communication function other than the second near field communication unit 251.

The determination unit 212 of the information processing device 12 includes an IMU (Inertial Measurement Unit) sensor 231, a wearing position determination unit 232, and a wearing position transmission unit 233.

The notification unit 214 of the information processing device 12 includes a display unit 271.

In the embodiment, near field communication is performed between the first near field communication unit 151 and the second near field communication unit 251.

Specifically, in the embodiment, the first near field communication unit 151 and the second near field communication unit 251 are set to a near field communication enabled state when they are close to each other within a predetermined distance. In the embodiment, the biological information measuring apparatus 11 and the information processing device 12 are regarded as being close to each other when the first near field communication unit 151 and the second near field communication unit 251 are close to each other.

In the embodiment, the first near field communication unit 151 and the second near field communication unit 251 can perform the detection of mutual closeness, power supply and power reception, and communication of information through the near field communication.

Note that the detection of closeness may be referred to as closeness determination, closeness estimation or the like, for example.

The information processing device 12 is described below.

The second near field communication unit 251 has a function to perform the near field communication. In the embodiment, this function includes a power supply function to wirelessly supply power.

The second near field communication unit 251 has a function to notify the wearing position determination unit 232 of the communication status.

In the embodiment, when near field communication is established between the second near field communication unit 251 and the first near field communication unit 151, the second near field communication unit 251 notifies information representing that fact to the wearing position determination unit 232. This information represents the fact that the first near field communication unit 151 and the second near field communication unit 251 become close to each other enough to enable near field communication, and in the embodiment, represents the fact that the biological information measuring apparatus 11 and the information processing device 12 become close to each other.

The IMU sensor 231 includes a triaxial acceleration sensor and a triaxial gyro sensor, for example.

In the embodiment, the IMU sensor 231 outputs a signal representing the acceleration and a signal representing the angular velocity based on the detection result of the acceleration sensor.

The IMU sensor 231 may include another sensor such as a temperature sensor, for example.

Note that another sensor, such as an acceleration sensor, may be used instead of the IMU sensor 231.

The wearing position determination unit 232 determines the wearing position of the biological information measuring apparatus 11 based on the detection result of the IMU sensor 231. In the embodiment, the wearing position determination unit 232 determines the wearing position of the biological information measuring apparatus 11 based on the detection result of the IMU sensor 231, and the communication status of the second near field communication unit 251.

Note that the determination of the wearing position may be referred to as wearing position detection, wearing position estimation or the like, for example.

In the embodiment, the wearing position is the part of the user 31 where the biological information measuring apparatus 11 is mounted.

Note that the wearing position of the biological information measuring apparatus 11 in the body may be referred to as wearing part or the like, for example.

As a specific example, the wearing position determination unit 232 detects the movement of the information processing device 12 based on the detection result of the IMU sensor 231, and determines the wearing position of the biological information measuring apparatus 11 based on the detection result of the movement.

Note that the detection of the movement may be referred to as movement determination, movement estimation or the like, for example.

As an example, the wearing position determination unit 232 may determine the wearing position of the biological information measuring apparatus 11 based on the distance between the position of a predetermined start point and the position of a predetermined end point regarding the position of the information processing device 12.

As another example, the wearing position determination unit 232 may determine the wearing position of the biological information measuring apparatus 11 based on the distance and direction of the position of the predetermined start point of and the position of the predetermined end point regarding the position of the information processing device 12. In this case, the wearing position determination unit 232 may use the relationship between the position and direction.

As the position of the predetermined start point, a position set in advance such as the front of the face or the front of the chest of the user 31 may be used, for example. In addition, as the position of the predetermined end point, a position where the information processing device 12 is close to the biological information measuring apparatus 11 is used, for example. This position may be a position where the near field communication between the information processing device 12 and the biological information measuring apparatus 11 is changed from the non-established state to the established state.

When the information related to the height of the user 31 is acquired by the second acquiring unit 213, the wearing position determination unit 232 may determine the wearing position of the biological information measuring apparatus 11 also based on that information, for example.

Note that the second acquiring unit 213 may notify information related to the height of the user 31 to the biological information measuring apparatus 11 through the second communication unit 211. The biological information measuring apparatus 11 may store the information related to the height of the user 31 notified from the information processing device 12. In addition, the biological information measuring apparatus 11 may use the information related to the height of the user 31 in algorithm computation at the algorithm computation unit 173.

The information related to the height of the user 31 may be input to the information processing device 12 by the user 31 operating the operation unit of the information processing device 12, for example. In this case, the second acquiring unit 213 acquires the information related to the height of the user 31 based on input information from the outside of the information processing device 12. Note that when the information processing device 12 is a smartphone, the operation unit may be the touch panel of the smartphone, for example.

As another example, the information related to the height of the user 31 may be acquired by moving the information processing device 12 from the position above the head to the position of the feet of the user 31 in a standing position, or by moving the information processing device 12 from the position of the feet to the position above the head of the user 31. In this case, the second acquiring unit 213 acquires the information for specifying the height of the user 31 on the assumption that the difference between the start point and the end point of the movement in the gravity direction is the height, based on the detection result of the IMU sensor 231.

The wearing position determination unit 232 has a function to display on the display unit 271 the information required for determining the wearing position, or the determination result of the wearing position.

The information required for determining the wearing position may be information of an instruction for the user 31 to determine the wearing position.

The wearing position transmission unit 233 transmits the determination result of the wearing position determination unit 232 to the first near field communication unit 151 of the biological information measuring apparatus 11 by means of the second near field communication unit 251.

The determination result is information representing the wearing position of the biological information measuring apparatus 11.

The display unit 271 includes a screen, and outputs and displays various information on the screen.

In the embodiment, the display unit 271 outputs and displays information from the wearing position determination unit 232, for example.

The biological information measuring apparatus 11 is described below.

The first near field communication unit 151 has a function to perform the near field communication. In the embodiment, this function includes a power reception function to wirelessly receive power.

The first near field communication unit 151 notifies the first acquiring unit 132 of information received from the second near field communication unit 251 of the information processing device 12.

This information is information representing the wearing position of the biological information measuring apparatus 11.

In the embodiment, when the biological information measuring apparatus 11 in the power-off state receives wireless power supply from the information processing device 12, it shifts to the power-on state.

As another example, the biological information measuring apparatus 11 may have the power-off state, an operating state, and a power saving state, which is an intermediate power consumption state between the power-off state and the operating state. Further, when the biological information measuring apparatus 11 in the power-off state or power saving state receives wireless power supply from the information processing device 12, it shifts to the operating state by using the power of the power supply, thus enabling the information communication between it and the information processing device 12. In this example, the operating state and the power saving state are the power-on state, for example.

The sensor unit 171 includes one or more sensors that detect information related to a living body. In the embodiment, the living body is the user 31.

Various sensors may be used as the sensor of the sensor unit 171. The information detected by the sensor may be various information.

As a specific example, an acceleration sensor or the like may be used as the sensor of the sensor unit 171, for example.

The wearing position setting holding part 172 holds, as the setting information of the wearing position, the information representing the wearing position of the biological information measuring apparatus 11 notified from the first near field communication unit 151.

The wearing position setting holding part 172 is composed with a storage unit such as a memory, and stores and holds the setting information of the wearing position, for example.

The algorithm computation unit 173 performs a predetermined algorithm computation related to biological information.

This algorithm computation computes the desired information based on the detection result of the sensor unit 171, for example.

In the embodiment, the algorithm computation unit 173 reflects, on the algorithm computation, a correction value corresponding to the setting information of the wearing position held by the wearing position setting holding part 172. As an example, this algorithm computation computes desired information based on the detection result of the sensor unit 171 and the wearing position. In this computation, the wearing position may be used to determine the threshold value in the algorithm computation, and in this case, the correction value is reflected on the threshold value, for example.

In the embodiment, the computation result of the algorithm computation unit 173 is an example of the biological information. In this case, the detection result of the sensor unit 171 may be or may not be regarded as the biological information.

As another example, the detection result of the sensor unit 171 may be regarded as the biological information. In this case, the algorithm computation of the algorithm computation unit 173 may be a computation of correcting the detection result of the sensor unit 171 based on the wearing position, and this correction result may be regarded as the biological information, for example.

In this manner, one or both of the computation result of the algorithm computation unit 173 and the detection result of the sensor unit 171 may be regarded as the biological information.

Note that it is possible to adopt a configuration in which no computation is performed for the detection result of the sensor unit 171. In this case, the algorithm computation unit 173 may not be provided in the first acquiring unit 132, and the correction based on the wearing position may not be performed. In this case, for example, the first acquiring unit 132 may acquire the detection result of the sensor unit 171 itself as biological information.

In addition, when the correction based on the wearing position is not performed, the wearing position setting holding part 172 may not be provided in the first acquiring unit 132.

The configuration example of the functional block illustrated in FIG. 2 is a specific configuration example of the functional block illustrated in FIG. 1, and each functional block illustrated in FIG. 1 does not necessarily have to be divided into more detailed functional blocks as illustrated in FIG. 2.

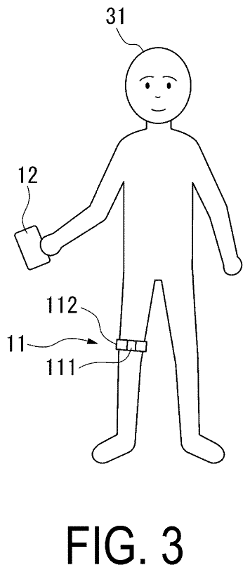

With reference to FIG. 3, an example of the wearing position of the biological information measuring apparatus 11 is described below.

FIG. 3 is a diagram illustrating an example of a state where the information processing system 1 according to the embodiment is held by the user 31.

In the example illustrated in FIG. 3, the biological information measuring apparatus 11 is mounted on the part around the right knee of the user 31.

In addition, in the example illustrated in FIG. 3, the user 31 is holding the information processing device 12 with the right hand of the user 31.

In the examples of FIGS. 1 to 3, the user 31 wears one biological information measuring apparatus 11, but the user 31 may wear two or more biological information measuring apparatuses 11 at different parts, for example.

In this case, each of the two or more biological information measuring apparatuses 11 may be identified by the identification information of the biological information measuring apparatus 11.

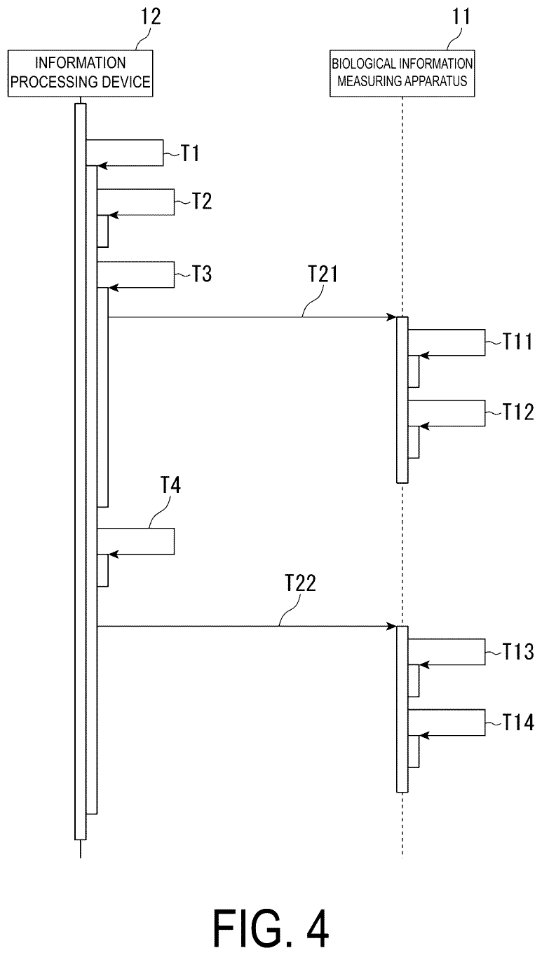

With reference to FIG. 4, an exemplary procedure of a process of determining the part where the biological information measuring apparatus 11 is mounted is described below.

FIG. 4 is a diagram illustrating a procedure of a process of determining the wearing part of the biological information measuring apparatus 11 according to the embodiment.

FIG. 4 schematically illustrates the biological information measuring apparatus 11 and the information processing device 12.

In this example, for convenience of description, the process performed by the information processing device 12 is referred to as process T1 to process T4, the process performed by the biological information measuring apparatus 11 is referred to as process T11 to process T14, and the communication process performed between the information processing device 12 and the biological information measuring apparatus 11 is referred to as process T21 to process T22.

This example assumes that the user 31 wears the biological information measuring apparatus 11 at a desired part of the user 31.

In addition, this example assumes that the user 31 is holding the biological information measuring apparatus 11 with the hand of the user 31.

In addition, in this example, the information processing device 12 has a function to wirelessly perform power supply to the biological information measuring apparatus 11 in the state where near field communication can be performed with the biological information measuring apparatus 11.

In addition, in this example, the biological information measuring apparatus 11 is initially in the power-off state. Further, the biological information measuring apparatus 11 has a function to shift to the power-on state when power is supplied from the information processing device 12 in the power-off state.

In the process T1, in the information processing device 12, the wearing position determination unit 232 starts the closeness detection operation of the biological information measuring apparatus 11.

In this example, the closeness detection operation is a series of operations performed by the information processing device 12 illustrated in FIG. 4.

The closeness detection operation at the information processing device 12 may be started in response to an operation of the user 31 on the operation unit, or started in response to the user 31 bringing the information processing device 12 close to the biological information measuring apparatus 11, for example.

Note that when the information processing device 12 is a smartphone, the operation unit may be the touch panel of the smartphone, for example. The user 31 may activate an application, a screen or the like for executing the closeness detection operation by operating the information processing device 12.

In addition, when the near field communication with the biological information measuring apparatus 11 is enabled in the state where the closeness detection operation is not started, the information processing device 12 may activate an application, a screen or the like for executing the closeness detection operation, for example. In this case, the user 31 can activate the closeness detection operation without operating the operation unit of the information processing device 12.

As a specific example, the user 31 causes the information processing device 12 to activate the activation screen of the closeness detection operation by bringing the information processing device 12 close to the biological information measuring apparatus 11. Thereafter, the user 31 brings the information processing device 12 to the position of the start point such as the front of the face of the user 31, and then brings the information processing device 12 to the position of the end point close to the biological information measuring apparatus 11. The information processing device 12 performs a process for determining the wearing position between the position of the start point and the position of the end point. Such a configuration is convenient for the user 31 because the application for performing the closeness detection operation can be activated, or the screen for performing the closeness detection operation can be activated in the application that has already been activated without operating the information processing device 12 with the finger of the user 31 or the like.

In the process T2, in the information processing device 12, the wearing position determination unit 232 detects the detection start position based on the detection result of the IMU sensor 231. Note that the detection start position may be referred to as the determination start position, or the position of the start point or the like, for example.

In the embodiment, the user 31 then moves the information processing device 12 to the position of the biological information measuring apparatus 11 with the information processing device 12 held with the hand of the user 31.

In the embodiment, the position where the user 31 should place the information processing device 12 is set in advance as the detection start position. This position is the position of the front of the face of the user 31, but this is not limitative, and may be various positions such as the position of the front of the chest of the user 31, for example.

In the process T3, in the information processing device 12, the IMU sensor 231 measures the information related to the movement of the information processing device 12.

In this example, the information includes at least the movement distance of the information processing device 12, and may further include the rotational direction of the information processing device 12, for example.

It is assumed that in the process T21, in the information processing device 12, the wearing position determination unit 232 has confirmed that the near field communication with the first near field communication unit 151 of the biological information measuring apparatus 11 is enabled by means of the second near field communication unit 251. In this case, power is supplied from the information processing device 12 to the biological information measuring apparatus 11 through the near field communication.

In the process T11, the biological information measuring apparatus 11 detects the power supply from the information processing device 12.

In the process T12, the biological information measuring apparatus 11 activates from the power-off state to the power-on state.

In the process T4, in the information processing device 12, the wearing position determination unit 232 determines the wearing position of the biological information measuring apparatus 11 based on the detection result of the IMU sensor 231.

The timing of the process T4 performed by the information processing device 12 does not necessarily have to be later than the timings the process T11 and the process T12 performed by the biological information measuring apparatus 11. Specifically, the process T4 performed by the information processing device 12 and the process T11 and the process T12 performed by the biological information measuring apparatus 11 may be performed independently of each other, for example.

In the process T22, in the information processing device 12, the wearing position determination unit 232 transmits information representing the setting details of the wearing position of the biological information measuring apparatus 11 to the biological information measuring apparatus 11 by means of the wearing position transmission unit 233.

In this example, this information is communicated by the near field communication of the second near field communication unit 251 of the information processing device 12 and the first near field communication unit 151 of the biological information measuring apparatus 11, but it may be communicated by other radio communication.

In the process T13, in the biological information measuring apparatus 11, the wearing position setting holding part 172 holds information representing the wearing position of the biological information measuring apparatus 11, as setting information of the wearing position based on the setting details of the wearing position received from the information processing device 12.

In the process T14, in the biological information measuring apparatus 11, the algorithm computation unit 173 applies setting information of the algorithm computation corresponding to the setting information of the wearing position.

The setting information of the algorithm computation may include parameter values such as threshold values in the algorithm computation, and may include the procedure of the algorithm computation, for example. In this example, the process of applying setting information of the algorithm computation corresponding to the setting information of the wearing position is an example of the process of reflecting, on the acquired biological information, the correction value corresponding to setting information of the wearing position.

In this manner, in the processing procedure illustrated in FIG. 4, the information processing device 12 held with the hand of the user 31 processes the signal from the IMU sensor 231 when moved in the direction of the biological information measuring apparatus 11 to calculate the distance, direction, angle or the like related to the movement with respect to the gravity direction and the like. The information processing device 12 refers to the signal from the IMU sensor 231 at least from the time it is moved by the user 31 until it comes close to the biological information measuring apparatus 11. The information processing device 12 determines the part of the body of the user 31 where the biological information measuring apparatus 11 is mounted based on the distance, direction or the like of the movement, and transmits the determined information representing the wearing position to the biological information measuring apparatus 11. The biological information measuring apparatus 11 sets the wearing position of its own apparatus based on the information received from the information processing device 12.

An example of an information notification from the information processing device 12 to the user 31 in the processing procedure illustrated in FIG. 4 is described below.

In this example, in the information processing device 12, the wearing position determination unit 232 makes an information notification for starting the closeness detection operation of the biological information measuring apparatus 11 to the user 31 in the process T1, and makes an information notification for indicating the setting result of the wearing position of the biological information measuring apparatus 11 in the process T22.

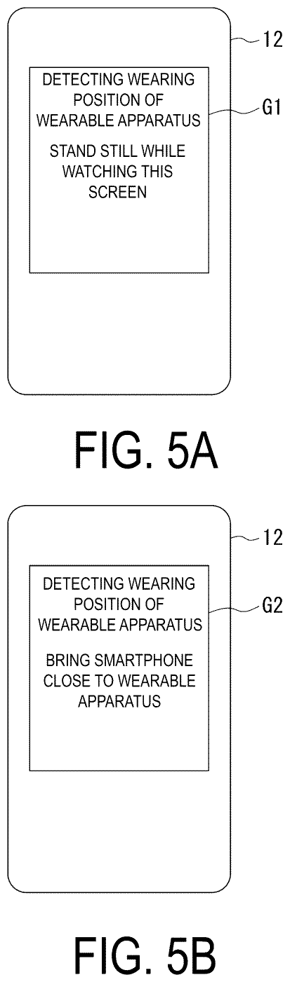

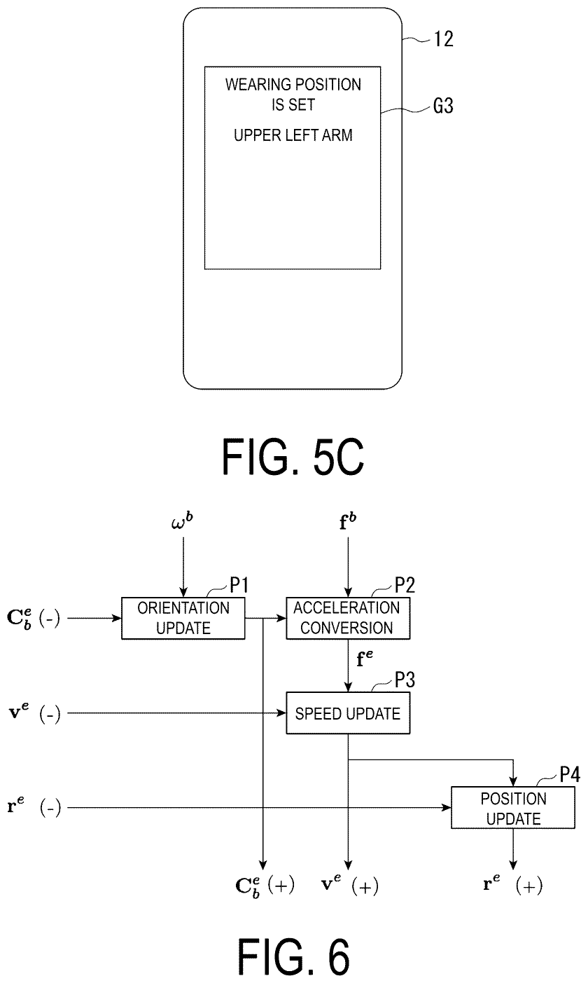

FIG. 5A is a diagram illustrating a first screen display G1, which is an example of a screen display of the information processing device 12 according to the embodiment.

In the information processing device 12, the wearing position determination unit 232 performs the first screen display G1 notifying the user 31 of information instructing to set the information processing device 12 to the initial position in the process T1.

In the example illustrated in FIG. 5A, the first screen display G1 indicates that the detection of the wearing position of the wearable apparatus is to be performed, and requests the user 31 to stand still while looking at the screen of the information processing device 12. In the embodiment, the biological information measuring apparatus 11 is an example of the wearable apparatus.

In this manner, the user 31 stands still and looks at the screen by setting the screen of the information processing device 12 at the position of the front of the face of the user 31 while holding the information processing device 12 with the hand of the user 31. In this example, this position is the initial position of the movement of the information processing device 12.

The user 31 can determine that the detection of the wearing position of the wearable apparatus is ready to start by looking at the content of the first screen display G1, for example.

FIG. 5B is a diagram illustrating a second screen display G2, which is an example of a screen display of the information processing device 12 according to the embodiment. In the information processing device 12, the wearing position determination unit 232 determines whether the information processing device 12 is set to a predetermined state based on the detection result of the IMU sensor 231 after performing the first screen display G1 in the process T1. Then, the wearing position determination unit 232 performs the second screen display G2 when determining that the information processing device 12 is set to the predetermined state.

This predetermined state is a state for regarding the state as a state where the user 31 is still and the screen of the information processing device 12 is set at the position of the front of the face of the user 31, and a given state may be set in advance. As a specific example, the predetermined state may include a state where the information processing device 12 is still or substantially still, and include a state where the screen of the information processing device 12 is parallel or substantially parallel to the gravity direction.

Note that as the state where the information processing device 12 is substantially still, a state where the movement speed of the information processing device 12 is smaller than a predetermined value may be used, for example.

In addition, as the state where the screen of the information processing device 12 is substantially parallel to the gravity direction, a state where a shift from the state where the screen of the information processing device 12 is parallel to the gravity direction is smaller than a predetermined angle may be used, for example.

In the example illustrated in FIG. 5B, the second screen display G2 indicates that the detection of the wearing position of the wearable apparatus is to be performed, and requests the user 31 to bring the smartphone close to the wearable apparatus. In the embodiment, the biological information measuring apparatus 11 is an example of the wearable apparatus, and the information processing device 12 is a smartphone as an example.

In this manner, the user 31 brings the information processing device 12 to the position close to the biological information measuring apparatus 11 with the information processing device 12 held with the hand of the user 31. In this example, this position is the arrival position of the movement of the information processing device 12.

The user 31 can determine that the movement of the information processing device 12 should be started by looking at the content of the second screen display G2.

FIG. 5C is a diagram illustrating a third screen display G3, which is an example of a screen display of the information processing device 12 according to the embodiment.

In the information processing device 12, the wearing position determination unit 232 performs the third screen display G3 in the process T22.

In the example illustrated in FIG. 5C, the third screen display G3 indicates that the wearing position of the wearable apparatus has been set, and that the wearing position is the upper left arm. In the embodiment, the biological information measuring apparatus 11 is an example of the wearable apparatus. Note that the upper left arm as the wearing position is an example, and changes depending on the actual wearing position of the wearable apparatus.

In this manner, the user 31 can confirm the completion of the determination and setting of the wearing position of the wearable apparatus, and can confirm the results of the determination and the setting.

Note that the information processing device 12 may transmit the setting details of the wearing position to the biological information measuring apparatus 11, and thereafter perform the third screen display G3 containing the applied setting details on the display unit 271, for example.

While the example illustrated in FIG. 5C is a case where the wearing position of the biological information measuring apparatus 11 is displayed in the form of letters, a configuration of displaying the wearing position by using a pattern, photograph or the like may be used as another example.

As a specific example, the wearing position determination unit 232 may display a pattern, photograph or the like of the human body, and further superimpose a pattern, photograph or the like of the biological information measuring apparatus 11 on the part corresponding to the wearing position in the displayed body. With such a display content, the user 31 can easily visually recognize the position where the biological information measuring apparatus 11 is mounted in the body. Further, for example, when the user 31 wears two or more biological information measuring apparatuses 11, the identification information of the biological information measuring apparatus 11 may be displayed at each wearing position of the biological information measuring apparatus 11.

In the embodiment, in response to the fact that the information processing device 12 and the biological information measuring apparatus 11 become a near field communication enabled state, the information processing device 12 notifies the determination result of the wearing position of the biological information measuring apparatus 11 to the biological information measuring apparatus 11 so as to perform the setting, but as another example, a confirmation process by the user 31 may be provided before this setting.

For example, in the process T4 and the like illustrated in FIG. 4, the wearing position determination unit 232 may determine the wearing position of the biological information measuring apparatus 11 in response to the fact that the information processing device 12 and the biological information measuring apparatus 11 become a near field communication enabled state, and may make the user 31 confirm whether to allow the determination result to be set. As an example, the wearing position determination unit 232 displays on the display unit 271 the determination result of the wearing position and an inquiry about to whether to allow the wearing position to be set, and sets the wearing position by notifying it to the biological information measuring apparatus 11 when receiving an instruction allowing for the setting of the wearing position through an operation of the user 31.

Note that when the information processing device 12 is a smartphone, the operation unit operated by the user 31 may be the touch panel of the smartphone, for example.

In the information processing device 12, the wearing position determination unit 232 may display the inquiry for the user 31 when determining that the user 31 has set the screen of the information processing device 12 at the position of the front of the face of the user 31, for example.

In the information processing device 12, the wearing position determination unit 232 displays the screen of the inquiry for the user 31 prior to the third screen display G3 illustrated in FIG. 5C, for example. Then, when the setting of the wearing position is allowed by the user 31, the wearing position determination unit 232 performs the setting of the wearing position and performs the third screen display G3 illustrated in FIG. 5C.

In this manner, the information processing device 12 may perform the setting of the wearing position when it is moved to the position where the user 31 looks at the screen of the information processing device 12 after it is brought close to the biological information measuring apparatus 11.

In this case, the information processing device 12 and the biological information measuring apparatus 11 are farther from each other in comparison with the closest situation, and therefore the notification of the wearing position from the information processing device 12 to the biological information measuring apparatus 11 may be performed by other wireless communication schemes than NFC such as Bluetooth (registered trademark) LE, for example.

The wearing position determination unit 232 may use any method to determine whether the user 31 has set the screen of the information processing device 12 at the position of the front of the face of the user 31.

As an example, the wearing position determination unit 232 may determine whether the user 31 has set the screen of the information processing device 12 at the position of the front of the face of the user 31 based on the orientation of the information processing device 12 and the like determined from the detection result of the IMU sensor 231. As a specific example, the wearing position determination unit 232 may start the operation of the wearing position detection when the information processing device 12 is still and the lower side of the screen of the information processing device 12 is in the gravity direction or substantially the gravity direction.

As another example, the wearing position determination unit 232 may determine that the user 31 have set the screen of the information processing device 12 at the position of the front of the face of the user 31 when face recognition is performed based on an image captured by a camera not illustrated in the drawing that captures the screen side of the information processing device 12, and the face of the user 31 is captured in the captured image. Note that the face recognition may determine whether the image is the face of the user 31 based on the face information of the user 31 stored in advance in the information processing device 12, or determine whether the image is a human face without determining whether it is the user 31, for example. As a specific example, the information processing device 12 may start the operation of the apparatus position detection in response to the fact that a human face has been successfully recognized based on the image captured by the camera.

An example of a notification of the identification information of the biological information measuring apparatus 11 is described below.

In the information processing device 12, the wearing position determination unit 232 may make a notification of the identification information of the biological information measuring apparatus 11 when making a notification of information such as the wearing position of the biological information measuring apparatus 11 in the form of a display or the like. In this case, the identification information is set in the biological information measuring apparatus 11, and for example, the biological information measuring apparatus 11 stores the identification information of its own apparatus.

When the information processing device 12 is moved and brought close to the biological information measuring apparatus 11, and the near field communication is performed between the information processing device 12 and the biological information measuring apparatus 11, the wearing position determination unit 232 receives the identification information of the biological information measuring apparatus 11. The identification information may be transmitted from the biological information measuring apparatus 11 to the information processing device 12 in response to a request to the biological information measuring apparatus 11 from the information processing device 12, or may be voluntarily transmitted from the biological information measuring apparatus 11 to the information processing device 12, for example.

Note that when a plurality of the biological information measuring apparatuses 11 is used, unique identification information may be assigned to each biological information measuring apparatus 11, for example. As a specific example, when the user 31 wears a plurality of the biological information measuring apparatuses 11, unique identification information is assigned to each biological information measuring apparatus 11.

The following describes an example of a calculation process for the orientation, speed and position using the IMU sensor 231.

FIG. 6 is a diagram schematically illustrating a calculation process for the orientation, speed and position using the IMU sensor 231 according to the embodiment.

FIG. 6 schematically illustrates processes such as an orientation update P1, an acceleration conversion P2, a speed update P3, and a position update P4.

In this example, the IMU sensor 231 detects an angular velocity @b and an acceleration fb. Then, the wearing position determination unit 232 performs the computation illustrated in FIG. 6 based on the detection result. Note that as another example, the computation illustrated in FIG. 6 may be performed at the IMU sensor 231.

In the orientation update P1, a rotation matrix C_{b}^{e} (−) and the angular velocity ω^{b} are input, and the rotation matrix C_{b}^{e} (−) is output as a calculation result.

In the acceleration conversion P2, the acceleration f^{b} and the output from the orientation update P1 are input, and an acceleration f^{e} is output as a calculation result.

In the speed update P3, a speed v^{e} (−) and the output from the acceleration conversion P2 are input, and a speed v^{e} (+) is output as a calculation result.

In the position update P4, a position r^{e} (−) and the output from the speed update P3 are input, and a position r^{e} (+) is output as a calculation result.

In the example illustrated in FIG. 6, the subscript b represents data of the IMU sensor axis system, and the subscript e represents data of the earth coordinate system. Data of the earth coordinate system corresponds to data from the perspective of a person standing on the ground. Note that the subscript b is an abbreviation of body, and the subscript e is an abbreviation of earth.

In addition, in the example illustrated in FIG. 6, the subscript (−) represents data before the update, and the subscript (+) represents data after the update.

In addition, the rotation matrix C_{b}^{e} (−) and the rotation matrix C_{b}^{e} (+) are rotation matrices from the b coordinate system to the e coordinate system, and are rotation matrices that indicate in angles the orientation in the coordinate system after the conversion. In addition, the update of the position is performed based on the movement distance and direction.

In this example, computational equations of the equation (1) to equation (3) are generally used. Note that the method of calculation of the orientation, speed and position using the output from the IMU sensor 231 is not limited, and publicly known methods may be used although the specific description thereof is omitted here.

With the equation (1), the orientation data is updated by multiplying a skew symmetric matrix Ω_{ib}^{b} of the angular velocity ω^{b} output from the IMU sensor 231 by the rotation matrix C_{b}^{e} and adding it, and subtracting the influence of the Earth's rotation.

Here, t represents time, Δt represents the amount of time variation, and I represents unit matrix.

In addition, C_{b}^{e} (t+Δt) represents data after the update and corresponds to C_{b}^{e} (+), and C_{b}^{e} (t) represents data before the update and corresponds to C_{b}^{e} (−).

In addition, subscript i is an abbreviation of inertial.

$$[ Equation 1 ]$$

$${C}_{b}^{e} ( t + Δ t ) = {C}_{b}^{e} ( t ) \cdot exp ( {Ω}_{ib}^{b} \cdot Δ t ) - \{ exp ( {Ω}_{ie}^{e} \cdot Δ t ) - I \} {C}_{b}^{e} ( t ) \qquad ( 1 )$$

As illustrated in FIG. 6, the acceleration f^{e} is calculated by multiplying the acceleration f^{b} output from the IMU sensor 231 by the rotation matrix C_{b}^{e}.

With the equation (2), the speed v^{e} is updated by adding the acceleration f^{e} and gravity g^{e} and subtracting the influence of the Earth's rotation.

$$[ Equation 2 ]$$

$${v}^{θ} ( t + Δ t ) = {v}^{θ} ( t ) + \{ {f}^{e} ( t + Δ t ) + {g}^{e} - 2 {Ω}_{ie}^{e} \cdot {v}^{e} ( t ) \} \cdot Δ t \qquad ( 2 )$$

With the equation (3), the position r^{e} is updated by adding the speed v^{e}.

$$[ Equation 3 ]$$

$${r}^{e} ( t + Δ t ) = {r}^{e} ( t ) + \{ {v}^{e} ( t ) + {v}^{e} ( t + Δ t ) \} \cdot ( Δ t / 2 ) \qquad ( 3 )$$

In the example illustrated in FIG. 6, based on the detection result of the IMU sensor 231, the movement distance, movement direction and the like of the information processing device 12 are calculated and the orientation of the information processing device 12 with respect to the gravity direction is determined. The speed is determined through the integral of the acceleration, and the distance is determined through the integral of the speed.

The orientation of the information processing device 12 can be determined by the roll angle and the pitch angle, for example. The initial orientation of the information processing device 12 is identified in the still period in which the information processing device 12 is not moving. Whether the period is the still period is determined by using the acceleration and angular velocity of the information processing device 12, and respective threshold values, for example.

For example, the initial position may be the origin of the coordinate axis or the like, and the initial speed may be 0.

Note that since the yaw angle cannot be calculated from the acceleration, the yaw angle may be 0. As another example, as the yaw angle, the azimuth angle of the geomagnetic sensor may be used.

In the still period, the bias of the angular velocity may be calculated. Theoretically, in the still period, the information processing device 12 does not rotate and therefore the angular velocity is 0. However, since errors may occur in practice, the average value of the angular velocity in the still period may be used as the bias of the angular velocity.

When the information processing device 12 becomes the still state, the information processing device 12 starts integration using the acceleration and angular velocity. For example, the orientation update may use the angular velocity to integrate the orientation.

By using the calculation process of this example, the information processing device 12 can calculate the movement distance and direction from the initial position as vector data, and can determine the wearing position of the biological information measuring apparatus 11 based on the distance and the direction.

Note that for example, a change in orientation with respect to the rotational direction and the gravity direction from the initial position of the information processing device 12 may be used as reference information for the determination of the wearing position.

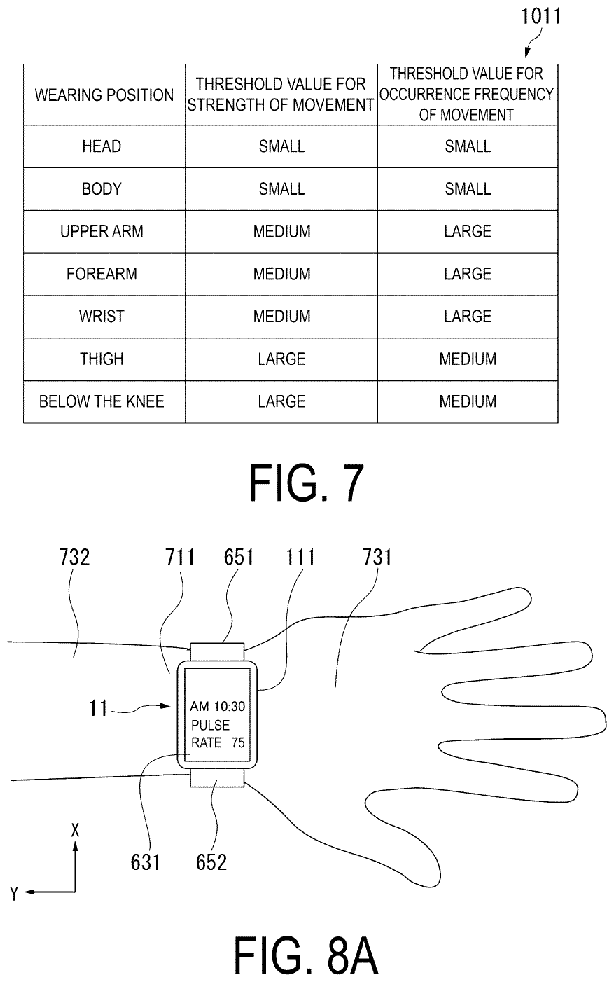

With reference to FIG. 7, a correction corresponding to the wearing position of the biological information measuring apparatus 11 is described below.

FIG. 7 is a diagram illustrating a table 1011 showing an example of a relationship between the threshold value and the wearing position of the biological information measuring apparatus 11 according to the embodiment.

Note that the example illustrated in FIG. 7 illustrates a table format for convenience of description, the table format does not necessarily have to be used.

In the example illustrated in FIG. 7, the table 1011 shows correspondence relationships between the wearing position, the threshold value for the strength of the movement, and the threshold value for the occurrence frequency of the movement.

As the threshold value for the strength of the movement, values of small, small, medium, medium, medium, and large are used for the head, body, upper arm, forearm, wrist, thigh, and below the knee, respectively as the wearing position.

As the threshold value for the occurrence frequency of the movement, values of small, small, large, large, large, medium, and medium are used for the head, body, upper arm, forearm, wrist, thigh, and below the knee, respectively as the wearing position.

Here, small, medium, and large are indicated in the ascending order from small to large of the threshold values. The small, medium and large in this example represent tendencies of the threshold values, not exact values. Specifically, two or more types of threshold values may be used for the threshold value corresponding to small, and the same applies to the medium and large.

In addition, the wearing positions shown in the table 1011 are examples, and this example is not limitative.

Note that tendencies of the sizes of threshold values corresponding to respective wearing positions do not necessarily have to be limited to the examples of the table 1011, and other configurations may be used.

In this manner, in the embodiment, the threshold value may be adjusted in accordance with the wearing position of the biological information measuring apparatus 11.

For example, in the biological information measuring apparatus 11, the information about the correspondence relationship shown in the table 1011 may be stored in the algorithm computation unit 173 of the first acquiring unit 132 and the like, and, when the algorithm computation includes the threshold value for the strength of the movement or the threshold value for the occurrence frequency of the movement, the algorithm computation unit 173 may adjust one or both of these threshold values in accordance with the wearing position. In this example, such an adjusting process corresponds to the process of reflecting, on the acquired biological information, the correction value corresponding to the wearing position.

In this example, since the strength of the movement indicated in the signal from the acceleration sensor is largest at the leg part such as the thigh and below the knee and smallest at the head and the body, the threshold values are changed in accordance with this fact.

In addition, in this example, since the occurrence frequency of the movement indicated in the signal from the acceleration sensor is smallest at the head and the body and highest at the arm such as the upper arm, forearm and wrist, the threshold values are changed in accordance with this fact.

Note that for example, regarding the arm, the threshold value may be more finely adjusted for two or more of the upper arm, forearm and wrist in consideration of the fact that the strength of the movement and the occurrence frequency of the movement increase in the order of the upper arm, forearm, and wrist.

Likewise, regarding the leg part, the threshold value may be changed between the thigh and below the knee.

In addition, for example, the threshold value may be adjusted by further by separating the ankle with respect to below the knee.

This example assumes that the strength of the movement of each part satisfies a predetermined condition when the value representing the strength of the movement of each part is equal to or greater than the threshold value of each part, and that the strength of the movement of each part does not satisfy the predetermined condition when the value representing the strength of the movement of each part is smaller than the threshold value of each part.

In addition, this example assumes that the occurrence frequency of the movement of each part satisfies a predetermined condition when the value representing the occurrence frequency of the movement of each part is equal to or greater than the threshold value of each part, and that the occurrence frequency of the movement of each part does not satisfy the predetermined condition when the value representing the occurrence frequency of the movement of each part is smaller than the threshold value of each part.

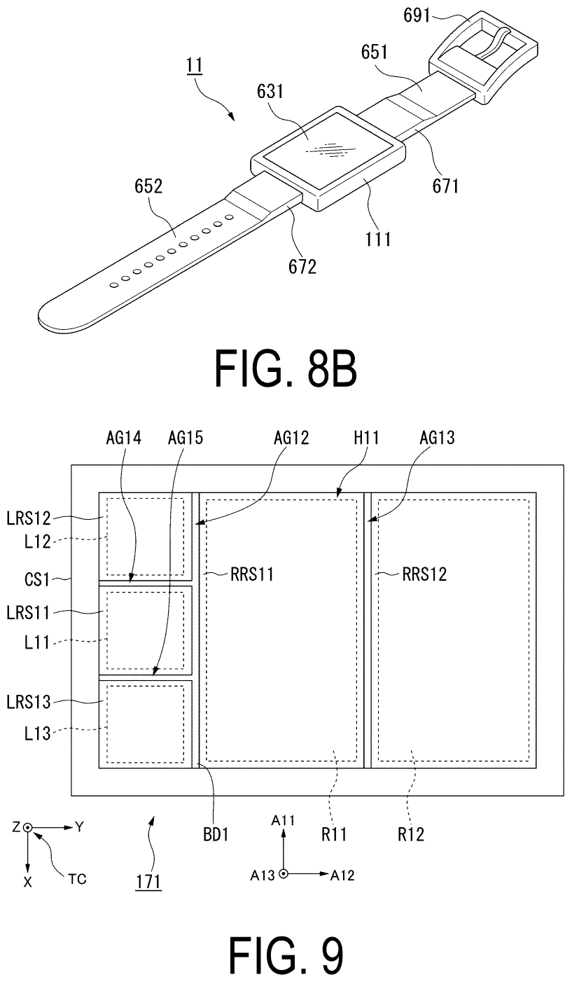

An example of the biological information measuring apparatus 11 of wristwatch type is described below with reference to FIGS. 8A and 8B.

FIG. 8A is a diagram illustrating an example of a state where the biological information measuring apparatus 11 of wristwatch type according to the embodiment is mounted.

FIG. 8B is a diagram illustrating an example of an external appearance of the biological information measuring apparatus 11 of wristwatch type according to the embodiment.

FIGS. 8A and 8B illustrate an external appearance example of the biological information measuring apparatus 11 that is configured as a wristwatch type apparatus provided with a function of a pulse rate meter.

In addition, FIG. 8A illustrates a state where the user 31 wears the biological information measuring apparatus 11 of wristwatch type at a wrist 711.

The biological information measuring apparatus 11 includes the body unit 111, a first band 651, a second band 652, a first extendable part 671, a second extendable part 672, and a coupling part 691 corresponding to a buckle.

The body unit 111 includes a display surface 631.

In the example of FIGS. 8A and 8B, the mounting unit 112 illustrated in FIG. 2 is composed of the first band 651, the second band 652, the first extendable part 671, the second extendable part 672, and the coupling part 691.

The first extendable part 671, the first band 651, and the coupling part 691 are coupled in series on one side of the body unit 111.

The second extendable part 672 and the second band 652 are coupled in series on the other side of the body unit 111.

As with wristwatches, it has a configuration in which the rod-shaped projecting portion of the coupling part 691 and one of a plurality of holes of the second band 652 are detachably engaged with each other.

In this example, the sensor unit 171 provided in the body unit 111 includes a pulse sensor that detects the pulse rate of the user 31. A sensor using a light detection may be used as the pulse sensor, for example.

In this example, the body unit 111 displays the acquired pulse rate and time on the display surface 631.

FIG. 8A illustrates the wrist 711, a hand 731, and a lower arm 732 of the user 31, and illustrates a state where the biological information measuring apparatus 11 of wristwatch type is mounted on the wrist 711.

In addition, FIG. 8A illustrates an XY orthogonal coordinate system that is a two-dimensional orthogonal coordinate system for convenience of description.

In the example illustrated in FIG. 8A, the direction of the circumferential length of the wrist 711 is along the X-axis direction, and the direction from the wrist 711 toward the lower arm 732 is along the Y-axis direction.

The first extendable part 671 and the second extendable part 672 are configured to be deformable in directions such as the X-axis direction and Y-axis direction, for example.

In this example, the biological information measuring apparatus 11 is configured as a wristwatch type wearable apparatus, but the biological information measuring apparatus 11 may be wearable apparatuses of other various forms.

The biological information measuring apparatus 11 may be configured as a wearable apparatus in the form of a hat or helmet mounted on head, a wearable apparatus in the form of eyeglasses or goggles mounted on the eyes of the head, a wearable apparatus in the form of a necklace mounted on the neck, a wearable apparatus in the form of a band or belt mounted on the waist, or a wearable apparatus in the form of a band or belt mounted on the upper arm, forearm, wrist, thigh, or below the knee, for example.

In this manner, the wearable apparatus may be mounted on various parts of the body of the user 31.

With reference to FIG. 9, a configuration example of the sensor unit 171 of the biological information measuring apparatus 11 is described below.

Note that the configuration of the sensor unit 171 illustrated in FIG. 9 is an example, and is not limitative, and, other various configurations may be used.

FIG. 9 is a diagram schematically illustrating a configuration example of the sensor unit 171 of the biological information measuring apparatus 11 according to the embodiment.

FIG. 9 illustrates an XYZ orthogonal coordinate system that is a three-dimensional orthogonal coordinate system TC for convenience of description.

In addition, FIG. 9 illustrates a first direction A11, a second direction A12 and a third direction A13 orthogonal to each other for convenience of description. The third direction A13 is the upward direction.

In the example illustrated in FIG. 9, as an example, the first direction A11 coincides with the negative direction of the X axis. In addition, in the example illustrated in FIG. 9, as an example, the second direction A12 coincides with the positive direction of the Y axis. In addition, in the example illustrated in FIG. 9, as an example, the third direction A13 coincides with the positive direction of the Z axis.

The sensor unit 171 includes a board BD1. In addition, the sensor unit 171 includes, on the board BD1, a first light emission part L11, a second light emission part L12, a third light emission part L13, a first light reception part R11, a second light reception part R12, a first light emission side optical member LRS 11, a second light emission side optical member LRS 12, a third light emission side optical member LRS 13, a first light reception side optical member RRS11, a second light reception side optical member RRS12, and a housing member CS1.

Note that the example of FIG. 9 illustrates a state of the inner surface on the upper side of the sensor unit 171, and omits a coating member disposed on the upper side of it.

In addition, FIG. 9 illustrates examples of voids, a twelfth void AG12, a thirteenth void AG13, a fourteenth void G14, and a fifteenth void G15.

In addition, FIG. 9 illustrates a first opening H11.

The board BD1 may be any board as long as it can be used as a board of the sensor unit 171. The board BD1 is a board using a phenol resin, a polyimide resin, a fluorine resin, or an epoxy resin, for example.

In this example, of the two surfaces of the board BD1, the surface on the positive direction side in the Z axis is referred to as the top surface of the board BD1, and the surface on the negative direction side in the Z axis is referred to as the bottom surface of the board BD1 for convenience of description.

The first light emission part L11 is a light-emitting element, light-emitting device or the like that emits light with a predetermined first wavelength band as first light. The first light emission part L11 is, for example, an LED (Light Emitting Diode), but may alternatively be another light-emitting element or another light-emitting device such as an OLED (Organic Light Emitting Diode), a u (Micro) LED, and a VCSEL (Vertical Cavity Surface Emitting Laser). In this example, as an example, a case where the first wavelength band is the green wavelength band is described. In this case, the first light emission part L11 emits green light as the first light.

The first light emission part L11 is provided on the top surface of the board BD1. Note that in this example, to avoid complicating the diagram, the member that couples the first light emission part L11 and the transmission path on the board BD1 is omitted. The member is, for example, a wire bonding, a coupling terminal or the like, but this is not limitative.

The first light reception part R11 is a light-receiving element, a light receiving device or the like that receives the first light emitted by the first light emission part L11. Note that the first light reception part R11 may be configured such that the first light can be received and that light with a wavelength band different from the first wavelength band cannot be received. The configuration that cannot receive light with a wavelength band different from the first wavelength band is achieved with a band pass filter or the like. In this example, as an example, the first light reception part R11 can receive the first light, and can receive light with a wavelength band different from the first wavelength band.

As viewed from the predetermined first direction A11 with respect to the board BD1, the first light reception part R11 is provided side by side with the first light emission part L11 in the second direction A12 on the top surface of the board BD1.

The first direction A11 may be any direction as long as it is parallel to the board BD1. In addition, the second direction A12 is a direction parallel to the board BD1 and orthogonal to the first direction A11.

Note that in the example illustrated in FIG. 9, to avoid complicating the diagram, the illustration of the member that couples the first light reception part R11 and the transmission path on the board BD1 is omitted. The member is, for example, a wire bonding, a coupling terminal or the like, but this is not limitative.

The first light emission side optical member LRS 11 transmits the first light. Note that the first light emission side optical member LRS 11 may be configured to transmit the first light and transmit light of a wavelength band different from the first wavelength band, or may be configured to transmit the first light and not transmit light of a wavelength band different from the first wavelength band. In this example, as an example, the first light emission side optical member LRS 11 transmits the first light, and transmits light of a wavelength band different from the first wavelength band. The first light emission side optical member LRS 11 is composed of a material with a refractive index of about 1.4 or greater that transmits the first light, and is, for example, a light-transmissive resin such as a transparent epoxy resin and a transparent acrylic resin, although this is not limitative. Note that the refractive index of a transparent epoxy resin is about 1.52. In addition, the refractive index of a transparent acrylic resin is about 1.45.

The first light emission side optical member LRS 11 covers the first light emission part L11 at the board BD1. In other words, the first light emission side optical member LRS 11 sandwiches and wraps the first light emission part L11 together with the board BD1. In this example, as an example, the first light emission side optical member LRS 11 is provided on the top surface of the board BD1 with no void formed between it and the first light emission part L11. In this case, no void is present between the first light emission side optical member LRS 11 and the first light emission part L11 except for the void unintentionally formed in the manufacturing process. In this manner, the sensor unit 171 can suppress refraction of the first light emitted from the first light emission part L11 between the first light emission side optical member LRS 11 and the board BD1. As a result, the optical design can be simplified in the sensor unit 171.

In addition, since the first light emission part L11 is covered with the first light emission side optical member LRS 11, the sensor unit 171 can suppress accidental touch of the user on the first light emission part L11, exposure of the first light emission part L11 to dust, moisture and the like. As a result, the sensor unit 171 can suppress occurrence of defects in the first light emission part L11.

Note that the sensor unit 171 may be configured such that a void is formed in a part between the first light emission side optical member LRS 11 and the first light emission part L11.

The first light reception side optical member RRS11 transmits the first light. Note that the first light reception side optical member RRS11 may be configured to transmit the first light and transmit light of a wavelength band different from the first wavelength band, or may be configured to transmit the first light and not transmit light of a wavelength band different from the first wavelength band. In this example, as an example, the first light reception side optical member RRS11 transmits the first light, and transmits light of a wavelength band different from the first wavelength band. The first light reception side optical member RRS11 is composed of a material with a refractive index of about 1.4 or greater that transmits the first light, and is, for example, a light-transmissive resin such as a transparent epoxy resin and a transparent acrylic resin, although this is not limitative. In this example, as an example, the resin making up the first light reception side optical member RRS11 is the same as the resin making up the first light emission side optical member LRS 11.

The first light reception side optical member RRS11 covers the first light reception part R11 at the board BD1. In other words, the first light reception side optical member RRS11 sandwiches and wraps the first light reception part R11 together with the board BD1. In this example, as an example, the first light reception side optical member RRS11 is provided on the top surface of the board BD1 with no void formed between it and the first light reception part R11. In this case, no void is present between the first light reception side optical member RRS11 and the first light reception part R11 except for the void unintentionally formed in the manufacturing process. In this manner, the sensor unit 171 can suppress refraction of the first light incident on the first light reception side optical member RRS11 between the first light reception side optical member RRS11 and the board BD1.

As a result, the optical design can be simplified in the sensor unit 171. In addition, since the first light reception part R11 is covered with the first light reception side optical member RRS11, the sensor unit 171 can suppresses accidental touch of the user 31 on the first light reception part R11, exposure of the first light reception part R11 to dust, moisture and the like. As a result, the sensor unit 171 can suppress occurrence of defects in the first light reception part R11.

Note that the sensor unit 171 may be configured such that a void is formed in a part between the first light reception side optical member RRS11 and the first light reception part R11.

The housing member CS1 is provided on the top surface of the board BD1. The housing member CS1 is a member making up the external shape of the sensor unit 171 together with the board BD1 and a coating member not illustrated in the drawing. In this example, as an example, the external shape of the housing member CS1 is a cuboid shape as a whole except for various openings formed in the housing member CS1, distortions due to manufacturing errors and the like for the purpose of simplifying the description. In this case, the top surface of the housing member CS1 is a surface parallel to the board BD1. Note that the top surface of the housing member CS1 may be a surface non-parallel to the board BD1. In addition, the height of the top surface of the housing member CS1 from the board BD1 is set to be greater than the height of the first light emission part L11 from the board BD1.

The housing member CS1 is composed of a highly light-reflective opaque resin, a transparent resin containing metal powder mixed therein, a metal or the like, for example. In this example, as an example, the housing member CS1 is a white epoxy resin that is substantially impervious to visible light and infrared light. In addition, in this example, the white epoxy resin is referred to as white resin for convenience of description.

The first opening H11 is formed in the housing member CS1.

The first opening H11 is a hole extending through the housing member CS1 in the third direction A13 intersecting the board BD1. In this example, as an example, of the two directions orthogonal to the board BD1, the third direction A13 is the direction from the board BD1 toward the first light emission part L11. In this case, the third direction A13 is orthogonal to the first direction A11 and the second direction A12 and coincides with the positive direction of the Z axis.

The coating member not illustrated in the drawing is a member provided at least at a part of the surface of the first light reception side optical member RRS11 on the side opposite to the surface in contact with the board BD1. The coating member is a member that sets the transmittance for the first light that is incident on the surface at an angle smaller than a first angle to a value greater than the transmittance for the first light that is incident on the surface at an angle equal to or greater than the first angle.

The coating member is, for example, an AR (Anti Reflection) coating, but may alternatively be another member that can set the transmittance for the first light that is incident on the surface at an angle smaller than a first angle to a value greater than the transmittance for the first light that is incident on the surface at an angle equal to or greater than the first angle. The first angle is, for example, an angle from 30 to 60 degrees, more preferably an angle of 42 degrees or greater.

In the example illustrated in FIG. 9, the coating member is provided at the top surfaces of the first light emission side optical member LRS 11, the second light emission side optical member LRS 12, the third light emission side optical member LRS 13, the first light reception side optical member RRS11, the second light reception side optical member RRS12, and the housing member CS1. Then, the coating members provided at the top surfaces may be partially or entirely coupled to or separated from each other.

Note that in the sensor unit 171, the coating member may not be provided in a part or all of the top surface of the first light emission side optical member LRS 11, a part or all of the top surface of the second light emission side optical member LRS 12, a part or all of the top surface of the third light emission side optical member LRS 13, a part or all of the top surface of the first light reception side optical member RRS11, a part or all of the top surface of the second light reception side optical member RRS12, or a part or all of the top surface of the housing member CS1.

The second light emission part L12 is provided side by side with the first light emission part L11 in the first direction A11 on the top surface of the board BD1. In the example illustrated in FIG. 9, the second light emission part L12 is provided next to the first light emission part L11 in the first direction A11. The second light emission part L12 is a light-emitting element, light-emitting device or the like that emits light with a predetermined second wavelength band as second light. The second light emission part L12 is, for example, an LED, but may be another light-emitting element or another light-emitting device such as an OLED, a uLED, and a VCSEL.

In this example, as an example, the second wavelength band is the red wavelength band. In this case, the second light emission part L12 emits red light as the second light. Note that in the example illustrated in FIG. 9, to avoid complicating the diagram, the illustration of the member that couples the second light emission part L12 and the transmission path on the board BD1 is omitted. The member is, for example, a wire bonding, a coupling terminal or the like, but this is not limitative.

The third light emission part L13 is provided side by side with the first light emission part L11 in the first direction A11 on the top surface of the board BD1. In the example illustrated in FIG. 9, the third light emission part L13 is provided next to the first light emission part L11 in the direction opposite to the first direction A11. That is, in this example, the first light emission part L11 is sandwiched by the second light emission part L12 and the third light emission part L13 in the first direction A11. The third light emission part L13 is a light-emitting element, light-emitting device or the like that emits light with a predetermined third wavelength band as third light. The third light emission part L13 is, for example, an LED, but may be another light-emitting element or another light-emitting device such as an OLED, a μLED, and a VCSEL. In this example, as an example, the third wavelength band is the wavelength band of the infrared ray. In this case, the third light emission part L13 emits infrared light as the third light. Note that in the example illustrated in FIG. 9, to avoid complicating the diagram, the illustration of the member that couples the third light emission part L13 and the transmission path on the board BD1 is omitted. The member is, for example, a wire bonding, a coupling terminal or the like, but this is not limitative.

The second light reception part R12 is provided side by side with the first light reception part R11 in the second direction A12 on the board BD1. In the example illustrated in FIG. 9, the second light reception part R12 is provided next to the first light reception part R11 in the second direction A12. The second light reception part R12 is a light-receiving element, a light receiving device or the like that receives the second light emitted by the second light emission part L12 and the third light emitted by the third light emission part L13.

Note that the second light reception part R12 may be configured such that the second light and third light can be received and that light with a wavelength band different from the second wavelength band and the third wavelength band can be received, or may be configured such that the second light and third light can be received and that light with a wavelength band different from the second wavelength band and the third wavelength band cannot be received. The configuration that cannot receive light with a wavelength band different from the second wavelength band and the third wavelength band is achieved with a band pass filter or the like, for example.

In this example, as an example, the second light reception part R12 can receive the second light and third light, and can receive light with a wavelength band different from the second wavelength band and the third wavelength band. Note that in the example illustrated in FIG. 9, to avoid complicating the diagram, the illustration of the member that couples the second light reception part R12 and the transmission path on the board BD1 is omitted. The member is, for example, a wire bonding, a coupling terminal or the like, but this is not limitative.

The second light emission side optical member LRS 12 transmits the second light. Note that the second light emission side optical member LRS 12 may be configured to transmit the second light and transmit a light with a wavelength band different from the second wavelength band, or may be configured to transmit the second light and not transmit light with a wavelength band different from the second wavelength band.

In this example, as an example, the second light emission side optical member LRS 12 transmits the second light and light with a wavelength band different from the second wavelength band. The second light emission side optical member LRS 12 is, for example, a light-transmissive resin such as a transparent epoxy resin and a transparent acrylic resin, although this is not limitative. In this example, as an example, the resin making up the second light emission side optical member LRS 12 is the same as the resin making up the first light emission side optical member LRS 11.

The second light emission side optical member LRS 12 covers the second light emission part L12 at the board BD1. In other words, the second light emission side optical member LRS 12 sandwiches and wraps the second light emission part L12 together with the board BD1. In this example, as an example, the second light emission side optical member LRS 12 is provided on the top surface of the board BD1 with no void formed between it and the second light emission part L12.

In this manner, for example, the same effects as those of the first light emission side optical member LRS 11 regarding the void can be achieved.

Note that the sensor unit 171 may be configured such that a void is formed in a part between the second light emission side optical member LRS 12 and the second light emission part L12.

The third light emission side optical member LRS 13 transmits the third light. Note that the third light emission side optical member LRS 13 may be configured to transmit the third light and transmit light with a wavelength band different from the third wavelength band, or may be configured to transmit the third light and not transmit light with a wavelength band different from the third wavelength band.

In this example, as an example, the third light emission side optical member LRS 13 transmits the third light and light with a wavelength band different from the third wavelength band. The third light emission side optical member LRS 13 is, for example, a light-transmissive resin such as a transparent epoxy resin and a transparent acrylic resin, although this is not limitative. In this example, as an example, the resin making up the third light emission side optical member LRS 13 is the same as the resin making up the first light emission side optical member LRS 11.

The third light emission side optical member LRS 13 covers the third light emission part L13 at the board BD1. In other words, the third light emission side optical member LRS 13 sandwiches and wraps the third light emission part L13 together with the board BD1. In this example, as an example, the third light emission side optical member LRS 13 is provided on the top surface of the board BD1 with no void formed between it and the third light emission part L13. In this case, no void is present between the third light emission side optical member LRS 13 and the third light emission part L13 except for the void unintentionally formed in the manufacturing process.

In this manner, for example, the same effects as those of the first light emission side optical member LRS 11 regarding the void can be achieved.

Note that the sensor unit 171 may be configured such that a void is formed in a part between the third light emission side optical member LRS 13 and the third light emission part L13.

The second light reception side optical member RRS12 transmits the second light and third light. Note that the second light reception side optical member RRS12 may be configured to transmit the second light and third light and light with a wavelength band different from the second wavelength band and the third wavelength band, or may be configured to transmit the second light and third light and not transmit light with a wavelength band different from the second wavelength band and the third wavelength band. In this example, as an example, the second light reception side optical member RRS12 transmits the second light and third light, and light with a wavelength band different from the second wavelength band and the third wavelength band. The second light reception side optical member RRS12 is, for example, a light-transmissive resin such as a transparent epoxy resin and a transparent acrylic resin, although this is not limitative. In this example, as an example, the resin making up the second light reception side optical member RRS12 is the same as the resin making up the first light emission side optical member LRS 11.

The second light reception side optical member RRS12 covers the second light reception part R12 at the board BD1. In other words, the second light reception side optical member RRS12 sandwiches and wraps the second light reception part R12 together with the board BD1. In this example, as an example, the second light reception side optical member RRS12 is provided on the top surface of the board BD1 with no void formed between it and the second light reception part R12.

In this manner, for example, the same effects as those of the first light reception side optical member RRS11 regarding the void can be achieved.

Note that the sensor unit 171 may be configured such that a void is formed in a part between the second light reception side optical member RRS12 and the second light reception part R12.

Use of information for specifying the dominant arm or the dominant leg is described below.

In the information processing device 12, the wearing position determination unit 232 may acquire the information for specifying the dominant arm of the user 31. In addition, the wearing position determination unit 232 may use the information for specifying the dominant arm of the user 31 when determining the wearing position of the biological information measuring apparatus 11. In addition, the wearing position determination unit 232 may notify the information for specifying the dominant arm of the user 31 to the biological information measuring apparatus 11 together with or separately from the information for specifying the wearing position.

The biological information measuring apparatus 11 may store the information for specifying the wearing position notified from the information processing device 12. In addition, the biological information measuring apparatus 11 may use the information for specifying the wearing position in algorithm computation of the algorithm computation unit 173.

The dominant arm is the right arm or left arm.

The information for specifying the dominant arm of the user 31 may be input to the information processing device 12 by the user 31 operating the operation unit of the information processing device 12, for example. In this case, the wearing position determination unit 232 acquires the information for specifying the dominant arm based on input information from the outside of the information processing device 12.

Note that when the information processing device 12 is a smartphone, the operation unit may be the touch panel of the smartphone, for example.

The process related to the dominant arm of the user 31 is described above, but a process related to the dominant leg of the user 31 may be performed together with or separately from that process, and the information for specifying the dominant leg may be used, for example.

Note that the process related to the dominant leg of the user 31 may be the same as the process related to the dominant arm of the user 31, and detailed description thereof is omitted, for example.

Use of information for specifying the left or right side of the body is described below.

As the information for specifying the wearing position of the biological information measuring apparatus 11, information for specifying either of the left and right sides of the body of the user 31 may be used, for example. In this example, the wearing position determination unit 232 of the information processing device 12 determines the wearing position.

Note that the left or right side of the body may be determined with respect to a reference line extending along the body height direction and passing through the center of the body width direction, or another reference may be used, for example. For example, the mounting on the right side or left side may be determined in accordance with a detection result about whether the information processing device 12 has made a right or left rotational movement with respect to an axis extending along the gravity direction.

Use of information for specifying the upper and lower sides of the body is described below.

Information for specifying the upper and lower sides of the body of the user 31 may be used as information for specifying the wearing position of the biological information measuring apparatus 11, for example. In this example, the wearing position determination unit 232 of the information processing device 12 determines the wearing position.

Note that the upper and lower sides of the body may be set with respect to a reference line extending along the body width direction and passing through the center of the body height direction, or another reference may be used, for example. Examples of the other reference may include the line of the height of the waist, the line of the height of the abdomen, the line of the height of the chest or the like.

For example, the wearing position may be determined from the head, body, arm, leg or the like based on the movement distance with respect to the gravity direction. In addition, the fact that the up-down direction of the information processing device 12 becomes opposite to the gravity direction when the information processing device 12 is held with the hand of the user 31 and the information processing device 12 is brought close to the feet may be used for the determination of the wearing position.

A display of an operating state of the wearable apparatus in the information processing device 12 is described below.

In the information processing device 12, the wearing position determination unit 232 may display the operating state of the biological information measuring apparatus 11 on the display unit 271 when setting the wearing position of the biological information measuring apparatus 11.

In this case, the wearing position determination unit 232 receives information about the operating state of the biological information measuring apparatus 11 when the near field communication is performed between the information processing device 12 and the biological information measuring apparatus 11 with the information processing device 12 moved and brought close to the biological information measuring apparatus 11. This information may be transmitted from the biological information measuring apparatus 11 to the information processing device 12 in response to a request to the biological information measuring apparatus 11 from the information processing device 12, or may be voluntarily transmitted from the biological information measuring apparatus 11 to the information processing device 12, for example.

For example, the operating state may be the remaining amount of the battery in the biological information measuring apparatus 11, and in this case, the biological information measuring apparatus 11 has a function to detect the remaining amount of the battery.

Confirmation of the wearing position of the wearable apparatus is described below.

The configuration in which when the near field communication between the information processing device 12 and the biological information measuring apparatus 11 is enabled when the information processing device 12 is moved by the user 31, that fact or information related to that fact is notified by the information processing device 12 to the user 31 may be used for confirmation of the wearing position of the biological information measuring apparatus 11, for example.

As a specific example, when the biological information measuring apparatus 11 is located inside the clothing mounted on the user 31, the user 31 can confirm the wearing position of the biological information measuring apparatus 11 by bringing the information processing device 12 close to the biological information measuring apparatus 11 through the clothing of the user 31. For example, with a configuration in which when the user 31 wears a plurality of the biological information measuring apparatuses 11 at different parts, the identification information of the biological information measuring apparatus 11 close to the information processing device 12 is notified to the user 31, the user 31 can determine whether the wearing positions of the plurality of biological information measuring apparatuses 11 are correct.

An example of the control of enabling/disabling the setting of the wearing position of the wearable apparatus is described below.

In the biological information measuring apparatus 11, the first near field communication unit 151 may perform a control of enabling the setting of the wearing position only in the state where near field communication can be established with the second near field communication unit 251 of the information processing device 12.

Note that this control may be performed by another component in the biological information measuring apparatus 11. The other component may be the wearing position setting holding part 172.

Another example of the control of enabling/disabling the setting of the wearing position of the wearable apparatus is described below.

The biological information measuring apparatus 11 may control of enabling the setting of the wearing position only in the state where the fact that the biological information measuring apparatus 11 is mounted on the body is detected.

Note that this control may be performed by any component in the biological information measuring apparatus 11. This component may be the algorithm computation unit 173 or the like, for example.

The state where the biological information measuring apparatus 11 is mounted the body is detected may be a state where a fact that the biological information measuring apparatus 11 is mounted on the skin of the human body is detected.

The sensor for detecting the fact that the biological information measuring apparatus 11 is mounted on the skin of the human body may be a skin contact sensor using an electrode for detecting the contact on the skin of the human body, or a pulse sensor for detecting closeness to the skin of the human body, for example.

An example of a process of determining the wearing position of the information processing device 12 based on the movement time is described below.

As an example other than the embodiment, in the information processing device 12, the wearing position determination unit 232 may determine the wearing position of the biological information measuring apparatus 11 based on the time taken for the information processing device 12 to move from the position of a predetermined start point to the position of a predetermined end point. For example, when the positions of the start point are the same, the time taken for the movement differs depending on whether the position of the end point is near the arm of the user 31 or near the feet of the user 31. Note that this example assumes that the speed of the information processing device 12 moved by the user 31 is constant or substantially constant. As the position of the start point, various positions such as the position of the front of the face of the user 31 may be used as in the embodiment.

In this manner, the information processing system 1 according to the embodiment includes the biological information measuring apparatus 11 and the information processing device 12.

The biological information measuring apparatus 11 includes the body unit 111, and the mounting unit 112 for mounting the body unit 111 on the user 31. In the body unit 111, the first communication unit 131 communicates with another apparatus, and the first acquiring unit 132 acquires the biological information of the user 31.

In the information processing device 12, the second communication unit 211 communicates with the biological information measuring apparatus 11, and the determination unit 212 determines the part where the biological information measuring apparatus 11 is mounted based on the movement distance from the start of the determination of the wearing position to the arrival at the biological information measuring apparatus 11.

Thus, the information processing system 1 according to the embodiment can specify the part of the body of the user 31 where the biological information measuring apparatus 11 as a wearable apparatus is actually attached, and set the wearing position corresponding to the part.

In addition, even when a plurality of the biological information measuring apparatuses 11 is mounted on the same the user 31, the information processing system 1 according to the embodiment can specify the part of the body of the user 31 where each biological information measuring apparatus 11 is actually attached, and set the wearing position. In this case, for example, with a configuration in which the information processing device 12 identifies the biological information measuring apparatus 11 based on the identification information, the correspondence relationship between each biological information measuring apparatus 11 and the wearing position can be determined.

In the information processing system 1 according to the embodiment, the wearing position of the biological information measuring apparatus 11 can be set in an easy manner for the user 31.

For example, in the related art, in some cases the operation for setting the part where the wearable apparatus is mounted on the body is cumbersome. More specifically, in the related art, in some cases it is necessary for the user to perform an operation of selecting the setting details by displaying the menu by operating buttons while looking at the screen of the wearable apparatus. In addition, in the related art, in some cases it is necessary for the user to perform an operation of selecting the wearing position by activating the setting screen with the application of the smartphone linked to the wearable apparatus.

In view of this, in the embodiment, the wearing position of the biological information measuring apparatus 11 can be set by a simple operation of the user 31 bringing the information processing device 12 close to the biological information measuring apparatus 11.

In the information processing system 1 according to the embodiment, the first acquiring unit 132 of the biological information measuring apparatus 11 may be configured to reflect, on the acquired biological information, a correction value corresponding to the determination result of the determination unit 212 of the information processing device 12. Note that such a configuration does not necessarily have to be used.

Thus, the information processing system 1 according to the embodiment can reflect the wearing position of the biological information measuring apparatus 11 on the biological information acquired by the biological information measuring apparatus 11.

In the information processing system 1 according to the embodiment, the first acquiring unit 132 of the biological information measuring apparatus 11 may be configured to reflect, on an algorithm for acquiring biological information, a correction value corresponding to the determination result of the determination unit 212 of the information processing device 12. Note that such a configuration does not necessarily have to be used.

Thus, the information processing system 1 according to the embodiment can reflect, on the wearing position of the biological information measuring apparatus 11, the biological information acquired by the biological information measuring apparatus 11.

Specifically, even when the same detection result is obtained by the same sensor, the biological information measuring apparatus 11 can obtain the processing result suitable for each wearing position of the biological information measuring apparatus 11 as a result of a process based on that detection result, for example. As a specific example, in the biological information measuring apparatus 11, in some cases it is preferable that the threshold value or the like for processing the detection result differ depending on the part where the biological information measuring apparatus 11 is mounted even when the same detection result is obtained by the same sensor.

For example, for a wearable apparatus that is wearable at a plurality of parts in the body, in some cases it is necessary to reflect, on the algorithm computation and the like, the difference in the strength of the movement, the occurrence frequency of the movement and the like of the human body depending on the wearing position.

In view of this, in the embodiment, when an algorithm computation is performed by using a sensor signal of an acceleration sensor and the like, the threshold value related to the body movement in the algorithm computation body and the like can be adjusted in accordance with the wearing position of the wearable apparatus, for example.

In the information processing system 1 according to the embodiment, the determination unit 212 of the information processing device 12 may be configured to detect the rotational direction of the information processing device 12 that is being moved, and determine the part where the biological information measuring apparatus 11 is mounted in accordance with the movement distance and the rotational direction of the information processing device 12. Note that such a configuration does not necessarily have to be used.

Thus, the information processing system 1 according to the embodiment can increase the accuracy of the determination of the wearing position of the biological information measuring apparatus 11 by referring to the movement distance, as well as the rotational direction, of the information processing device 12.

In the information processing system 1 according to the embodiment, the second acquiring unit 213 of the information processing device 12 acquires the information related to the height of the user 31. The determination unit 212 of the information processing device 12 may be configured to determine the part where the biological information measuring apparatus 11 is mounted based on the height of the user 31, the movement distance and the rotational direction. Note that such a configuration does not necessarily have to be used.

Thus, the information processing system 1 according to the embodiment can increase the accuracy of the determination of the wearing position of the biological information measuring apparatus 11 by referring also to the height of the user 31. For example, the determination unit 212 can increase the accuracy of the determination of the wearing position of the biological information measuring apparatus 11 by reducing the influence of individual differences related to the movement distance by referring to the height of the user 31.

Note that as another configuration example, the determination unit 212 of the information processing device 12 may be configured to determine the part where the biological information measuring apparatus 11 is mounted based on the height of the user 31 and the movement distance. Note that such a configuration does not necessarily have to be used.

In the information processing system 1 according to the embodiment, the first communication unit 131 of the biological information measuring apparatus 11 and the second communication unit 211 of the information processing device 12 perform the short-range wireless communication. The determination unit 212 of the information processing device 12 may be configured to determine arrival at the biological information measuring apparatus 11 through the short-range wireless communication performed by the first communication unit 131 and the second communication unit 211. Note that such a configuration does not necessarily have to be used.

Thus, the information processing system 1 according to the embodiment can determine the arrival of the information processing device 12 at the biological information measuring apparatus 11 based on establishment of short-range wireless communication between the information processing device 12 and the biological information measuring apparatus 11, which is convenient.

In the information processing system 1 according to the embodiment, the determination unit 212 of the information processing device 12 may be configured to determine that the part where the biological information measuring apparatus 11 is mounted is an arm when the movement distance is smaller than a predetermined distance, and determine that the part where the biological information measuring apparatus 11 is mounted is below the knee when the movement distance is equal to or greater than the predetermined distance. Note that such a configuration does not necessarily have to be used.

Thus, the information processing system 1 according to the embodiment can determine whether the wearing position of the biological information measuring apparatus 11 is the arm or below the knee in accordance with the value of the movement distance of the information processing device 12.

In the information processing system 1 according to the embodiment, regarding the first threshold value and the second threshold value that are correction values, the first acquiring unit 132 of the biological information measuring apparatus 11 may be configured to set the first threshold value for the strength of the movement in the case where the part where the biological information measuring apparatus 11 is mounted is an arm to a value smaller than the second threshold value for the strength of the movement in the case where the part where the biological information measuring apparatus 11 is mounted is below the knee. Note that such a configuration does not necessarily have to be used.

Thus, the information processing system 1 according to the embodiment can set a suitable threshold value for the strength of the movement of each part depending on whether the wearing position of the biological information measuring apparatus 11 is the arm or below the knee.

In the information processing system 1 according to the embodiment, regarding the third threshold value and the fourth threshold value that are correction values, the first acquiring unit 132 of the biological information measuring apparatus 11 may be configured to set the third threshold value for the occurrence frequency of the movement in the case where the part where the biological information measuring apparatus 11 is mounted is an arm to a value greater than the fourth threshold value for the occurrence frequency of the movement in the case where the part where the biological information measuring apparatus 11 is mounted is below the knee. Note that such a configuration does not necessarily have to be used.

Thus, the information processing system 1 according to the embodiment can set a suitable threshold value for the occurrence frequency of the movement of each part depending on whether the wearing position of the biological information measuring apparatus 11 is the arm or below the knee.

In the information processing system 1 according to the embodiment, the notification unit 214 of the information processing device 12 may be configured to make a notification of the part where the biological information measuring apparatus 11 is mounted determined by the determination unit 212. Note that such a configuration does not necessarily have to be used.

Thus, the information processing system 1 according to the embodiment can notify the user 31 of the wearing position of the biological information measuring apparatus 11. In this manner, the user 31 and the like can confirm the details of the notification.

A program for realizing the function of any constituent unit in any device described above may be recorded on a computer-readable recording medium, and the program may be read and executed by a computer system. The “computer system” as used here is assumed to include hardware such as an operating system (OS) or peripheral devices. The “computer-readable recording medium” is a storage device such as a portable medium such as a flexible disk, a magneto-optical disk, a read only memory (ROM), a compact disc (CD)-ROM, and a hard disk built into a computer system. The “computer-readable recording medium” is assumed to include a medium that holds a program for a certain period of time, such as a volatile memory provided inside of a computer system serving as a server or a client when the program is transmitted via a network such as the Internet or a communication line such as a telephone line. The volatile memory may be a RAM. The recording medium may be a non-transitory recording medium.

The above program may be transmitted from a computer system storing the program in a storage device or the like via a transmission medium or using transmission waves in a transmission medium to another computer system. The “transmission medium” that transmits the program refers to a medium having a function of transmitting information, such as a network such as the Internet or a communication line such as a telephone line.

The above program may be for realizing some of the above-described functions. The above program may be a so-called difference file, which can realize the above-described functions in combination with a program already recorded in the computer system. The difference file may be called a difference program.

The function of any constituent unit in any device described above may be realized by a processor. Each processing in the embodiment may be realized by a processor that operates based on information such as a program and a computer-readable recording medium that stores information such as a program. In the processor, the function of each unit may be realized by individual hardware, or the function of each unit may be realized by integrated hardware. The processor may include hardware, and the hardware may include at least one of a circuit for processing digital signals and a circuit for processing analog signals. The processor may be configured using one or both of one or more circuit devices or one or more circuit elements mounted on a circuit board. For the circuit device, an integrated circuit (IC) or the like may be used, and for the circuit element, a resistor, a capacitor, or the like may be used.

The processor may be a CPU (Central Processing Unit). However, the processor is not limited to the CPU, and various processors such as a graphics processing unit (GPU) or a digital signal processor (DSP) may be used. The processor may be a hardware circuit using an application-specific integrated circuit (ASIC). The processor may be configured by a plurality of CPUs or may be configured by a hardware circuit including a plurality of ASICs. The processor may be configured by a combination of a plurality of CPUs and a hardware circuit including a plurality of ASICs. The processor may include one or more of an amplifier circuit, a filter circuit, and the like for processing analog signals.

Although the embodiment has been described in detail with reference to the drawings, specific configurations are not limited to this embodiment and include designs and the like without departing from the gist of the present disclosure.

### Appendix

Configuration Example 1 to Configuration Example 12 are described below.

### Configuration Example 1

An information processing system includes a biological information measuring apparatus including a body unit including a first communication unit configured to communicate with another apparatus, and a first acquiring unit configured to acquire biological information of a user, and a mounting unit configured to mount the body unit on the user, and an information processing device including a second communication unit configured to communicate with the biological information measuring apparatus, and a determination unit configured to determine a part where the biological information measuring apparatus is mounted based on a movement distance from start of determination to arrival at the biological information measuring apparatus.

### Configuration Example 2

Configuration Example 1, in which the first acquiring unit of the biological information measuring apparatus reflects, on the biological information acquired, a correction value corresponding to a determination result of the determination unit of the information processing device.

### Configuration Example 3

The information processing system according to Configuration Example 1, in which the first acquiring unit of the biological information measuring apparatus reflects, on an algorithm for acquiring the biological information, a correction value corresponding to a determination result of the determination unit of the information processing device.

### Configuration Example 4

The information processing system according to any one of Configuration Examples 1 to 3, in which the determination unit of the information processing device detects a rotational direction of the information processing device that is being moved, and determines the part where the biological information measuring apparatus is mounted based on the movement distance and the rotational direction of the information processing device.

### Configuration Example 5

The information processing system according to Configuration Example 4, in which the information processing device includes a second acquiring unit configured to acquire information related to a height of the user, and the determination unit of the information processing device determines the part where the biological information measuring apparatus is mounted based on the height of the user, the movement distance and the rotational direction.

Note that the following configuration may be adopted. Modification A according to any one of Configuration Examples 1 to 3, in which the information processing device includes a second acquiring unit configured to acquire information related to a height of the user, and the determination unit of the information processing device determines the part where the biological information measuring apparatus is mounted based on the height of the user and the movement distance.

### Configuration Example 6

The information processing system according to any one of Configuration Examples 1 to 5, in which the first communication unit of the biological information measuring apparatus and the second communication unit of the information processing device perform a short-range wireless communication, and the determination unit of the information processing device determines arrival at the biological information measuring apparatus through the short-range wireless communication performed by the first communication unit and the second communication unit.

Note that Configuration Example 6 may depend on Modification A.

### Configuration Example 7

The information processing system according to any one of Configuration Examples 1 to 6, in which the determination unit of the information processing device determines that the part where the biological information measuring apparatus is mounted is an arm when the movement distance is smaller than a predetermined distance, and determines that the part where the biological information measuring apparatus is mounted is below the knee when the movement distance is equal to or greater than the predetermined distance.

Note that Configuration Example 7 may depend on Modification A.

### Configuration Example 8

Configuration Example 2, Configuration Example 3, the configuration example including Configuration Example 2, or the configuration example including Configuration Example 3, in which regarding a first threshold value and a second threshold value that are the correction value, the first acquiring unit of the biological information measuring apparatus sets the first threshold value for a strength of a movement in a case where the part where the biological information measuring apparatus is mounted is the arm to a value smaller than the second threshold value for a strength of a movement in a case where the part where the biological information measuring apparatus is mounted is below the knee.

The configuration example including Configuration Example 2 is any one of Configuration Example 4 to Configuration Example 7 including Configuration Example 2, for example. In addition, the configuration example including Configuration Example 3 is any one of Configuration Example 4 to Configuration Example 7 including Configuration Example 3, for example.

Note that Configuration Example 8 may depend from Modification A including Configuration Example 2, or Modification A including Configuration Example 3.

### Configuration Example 9

The information processing system according to Configuration Example 2, Configuration Example 3, the configuration example including Configuration Example 2, or the configuration example including Configuration Example 3, in which regarding a third threshold value and a fourth threshold value that are the correction value, the first acquiring unit of the biological information measuring apparatus sets the third threshold value for an occurrence frequency of the movement in the case where the part where the biological information measuring apparatus is mounted is the arm to a value greater than the fourth threshold value for an occurrence frequency of the movement in the case where the part where the biological information measuring apparatus is mounted is below the knee.

The configuration example including Configuration Example 2 is any one of Configuration Example 4 to Configuration Example 8 including Configuration Example 2, for example. In addition, the configuration example including Configuration Example 3 is any one of Configuration Example 4 to Configuration Example 8 including Configuration Example 3, for example.

Note that Configuration Example 9 may depend from Modification A including Configuration Example 2, or Modification A including Configuration Example 3.

### Configuration Example 10

The information processing system according to any one of Configuration Examples 1 to 9, in which the information processing device includes a notification unit configured to make a notification of the part where the biological information measuring apparatus is mounted determined by the determination unit.

It is also possible to provide an information processing device in the above-described information processing system.

### Configuration Example 11

An information processing device includes a communication unit configured to communicate with a biological information measuring apparatus configured to acquire biological information of a user, and a determination unit configured to determine a part where the biological information measuring apparatus is mounted based on a movement distance from start of determination to arrival at the biological information measuring apparatus.

It is also possible to provide a program recording medium that records a program executed by the processor in the above-described information processing device.

### Configuration Example 12

A non-transitory computer-readable storage medium configured to record a program, the program causing a computer to implement a communication function to communicate with a biological information measuring apparatus configured to acquire biological information of a user, and a determination function to determine a part where the biological information measuring apparatus is mounted based on a movement distance from start of determination to arrival at the biological information measuring apparatus.

What is claimed is:

1. An information processing system comprising:
a biological information measuring apparatus that includes a body unit and a mounting unit, the body unit including a first communication unit configured to communicate with another apparatus and a first acquiring unit configured to acquire biological information of a user, and the mounting unit configured to mount the body unit on the user; and
an information processing device that includes a second communication unit and a determination unit, the communication unit configured to communicate with the biological information measuring apparatus, and the determination unit configured to determine a part where the biological information measuring apparatus is mounted based on a movement distance from start of determination to arrival at the biological information measuring apparatus.

2. The information processing system according to claim 1, wherein the first acquiring unit of the biological information measuring apparatus reflects, on the biological information acquired, a correction value corresponding to a determination result of the determination unit of the information processing device.

3. The information processing system according to claim 1, wherein the first acquiring unit of the biological information measuring apparatus reflects, on an algorithm for acquiring the biological information, a correction value corresponding to a determination result of the determination unit of the information processing device.

4. The information processing system according to claim 2, wherein the determination unit of the information processing device detects a rotational direction of the information processing device that is being moved, and determines the part where the biological information measuring apparatus is mounted based on the movement distance and the rotational direction of the information processing device.

5. The information processing system according to claim 4, wherein
the information processing device includes a second acquiring unit configured to acquire information related to a height of the user, and
the determination unit of the information processing device determines the part where the biological information measuring apparatus is mounted based on the height of the user, the movement distance and the rotational direction.

6. The information processing system according to claim 2, wherein
the first communication unit of the biological information measuring apparatus and the second communication unit of the information processing device perform a short-range wireless communication, and
the determination unit of the information processing device determines arrival at the biological information measuring apparatus through the short-range wireless communication performed by the first communication unit and the second communication unit.

7. The information processing system according to claim 2, wherein the determination unit of the information processing device determines that the part where the biological information measuring apparatus is mounted is an arm when the movement distance is smaller than a predetermined distance, and determines that the part where the biological information measuring apparatus is mounted is below the knee when the movement distance is equal to or greater than the predetermined distance.

8. The information processing system according to claim 7, wherein regarding a first threshold value and a second threshold value that are the correction value,
the first acquiring unit of the biological information measuring apparatus sets the first threshold value for a strength of a movement in a case where the part where the biological information measuring apparatus is mounted is the arm to a value smaller than the second threshold value for a strength of a movement in a case where the part where the biological information measuring apparatus is mounted is below the knee.

9. The information processing system according to claim 8, wherein regarding a third threshold value and a fourth threshold value that are the correction value,
the first acquiring unit of the biological information measuring apparatus sets the third threshold value for an occurrence frequency of the movement in the case where the part where the biological information measuring apparatus is mounted is the arm to a value greater than the fourth threshold value for an occurrence frequency of the movement in the case where the part where the biological information measuring apparatus is mounted is below the knee.

10. The information processing system according to claim 2, wherein the information processing device includes a notification unit configured to make a notification of the part where the biological information measuring apparatus is mounted determined by the determination unit.

11. An information processing device comprising:
a communication unit configured to communicate with a biological information measuring apparatus configured to acquire biological information of a user; and
a determination unit configured to determine a part where the biological information measuring apparatus is mounted based on a movement distance from start of determination to arrival at the biological information measuring apparatus.

12. A non-transitory computer-readable storage medium configured to record a program, the program causing a computer to implement
a communication function to communicate with a biological information measuring apparatus configured to acquire biological information of a user; and
a determination function to determine a part where the biological information measuring apparatus is mounted based on a movement distance from start of determination to arrival at the biological information measuring apparatus.
